(19) United States
(12) Patent Application Publication
(10) Pub. No.: US 2022/0333070 A1
(43) Pub. Date: Oct. 20, 2022

(54) INDUCTION OF FUNCTIONAL ASTROCYTES FROM PLURIPOTENT STEM CELLS

(71) Applicant: LU License AB, Lund (SE)

(72) Inventors: Henrik Ahlenius, Lund (SE); Isaac Montferrer, Lund (SE)

(21) Appl. No.: 17/635,675

(22) PCT Filed: Aug. 16, 2019
(86) PCT No.: PCT/EP2019/072065

§ 371 Date: Feb. 15, 2022

(51) Int. Cl. C12N 5/079; C12N 15/86; C12N 15/63

(52) U.S. Cl. CPC C12N 5/0622; C12N 15/86; C12N 15/635; C12N 2506/02; C12N 2740/15042; C12N 2820/002; C12N 2501/115; C12N 2501/13; C12N 2501/155; C12N 2500/32; C12N 2501/999

(57) ABSTRACT

The present specification provides a method of producing induced functional astrocytes (iAs) from human pluripotent stem cells substantially more rapidly than previously achieved. These iAs express biomarkers and have functional characteristics typical of natural astrocytes. The iAs are useful in the exploration of astrocyte biology, pathophysiology, and in models of neurologic diseases and disorders.

### BACKGROUND

The derivation of astrocytes from human pluripotent stem cells is currently slow and inefficient. Astrocytes are known to carry out functions that are essential for normal brain physiology. However, much of this knowledge is derived from studies of mouse astrocytes, which differ in complexity from human astrocytes. Current protocols for generating human astrocytes are time-consuming—with durations on the order of many weeks or months—and technically challenging, and the resulting cells are incompletely characterized with respect to function.

### SUMMARY

Herein disclosed is a new transcription-factor-based reprogramming protocol to rapidly and efficiently generate functional induced astrocytes (iAs) from human pluripotent stem cells (hPSC). Overexpression of the transcription factor(s) nuclear factor 1 B-type (Nfib), or sex-determining region Y-box 9 (Sox9) and Nfib, in human pluripotent stem cells rapidly and efficiently yields homogeneous populations of induced astrocytes. In further embodiments, Nfia is used instead of, or in addition to, Nfib. The induced astrocytes exhibited molecular and functional properties resembling those of adult human astrocytes. Thus disclosed embodiments include methods of establishing in vitro and in vivo models for the study of astrocyte biology and neural and neurologic physiology and pathophysiology. In some embodiments, the hPSC is a human embryonic stem cell (hESC). In other embodiments, the hPSC is a human induced pluripotent stem cell (hiPSC).

Further disclosed are a population of isolated iAs generated through overexpression the transcription factor Nfib and/or Nfia each alone or together, or further in combination with Sox9 in human pluripotent stem cells. These iAs are useful in the study of astrocyte biology and the modeling of neural or neurologic diseases, both in vitro, and in vivo, for example, by intracerebral implant. In various embodiments at least 85%, 90%, or 95% of the cells in the population of iAs generated with selection are positive for one or more astrocyte biomarkers. In various aspects of these embodiments the positive biomarker is S100 calcium-binding protein B (S100B), Glial fibrillary acidic protein (GFAP), vimentin (VIM), Aldehyde Dehydrogenase 1 Family Member L1 (ALDH1L1), glutamate aspartate transporter (GLAST), CD44, Kir4,1, or any combination thereof. In some embodiments the iAs are positive for only a subset of these biomarkers to the exclusion of the assessment of others of the above listed biomarkers. In some embodiments the iAs are positive for only a subset of these biomarkers to the exclusion of the assessment of any other biomarkers. In some embodiments the iAs are positive for one, some, or all of S100B, GFAP, VIM, ALDH1L1, GLAST, CD44, Kir4.1, and one or more additional astrocyte biomarker. In some embodiments assessment of one or more additional astrocyte biomarkers is excluded. In aspects of these embodiments the additional astrocyte biomarker is nuclear factor 1 A-type (Nfia), glutamate transporter 1 (GLT-1), aldolase C (ALDOC), connexin 43 (CX43) also known as Gap junction alpha-1 protein (GJA1), hepatocyte cell adhesion molecule (HEPACAM), glutamate-ammonia Ligase (GLUL), tubulin, alpha 1A (TUBA1A), aquaporin-4 (AQP4), fatty acid binding protein 7 (FABP7), Sox9, or any combination thereof. In some embodiments the astrocyte biomarkers are S100B, GFAP, VIM, or any combination thereof. In further embodiments the population of isolated iAs do not express (or express only at very low levels) pluripotency, neural stem cell, neuronal, or oligodendrocyte genes.

In further embodiments the population of isolated iAs display one or more functional characteristics of astrocytes. In aspects of these embodiments the functional characteristic is having glycogen granules, glutamate uptake, ability to increase intracellular calcium levels (spontaneous or stimulated, for example, by ATP), support of synapse formation (for example, in cultures of induced neurons), an ability to increase cytokine expression in response to interleukin-1β (IL-1β) (e.g., IL-6, C-X-C motif chemokine ligand 8 (CXCL8), CXCL10, and/or C-C Motif Chemokine Ligand 5 (CCL5)), and formation of gap junctions.

In some embodiments, the isolated iAs are capable or surviving intracerebral implant. In aspects of these embodiments, the implanted iAs continue to express astrocyte biomarkers. In further aspects of these embodiments, the implanted iAs produce astrocytic processes that co-localize with synaptic structures on host neurons, for example, Bassoon⁺ and/or postsynaptic density-95⁺ (PSD95)⁺ synaptic structures. In still further aspects, the implanted iAs form gap-junctions with host cells.

In some embodiments, the isolated iAs are genetically modified to have a genetic lesion associated with a neural or neurologic disease or disorder. In some embodiments human pluripotent stem cells (e.g., hESC or iPSCs) are genetically modified to have a genetic lesion associated with a neurologic disease or disorder, and then treated to produce iAs bearing the genetic modification. In some embodiments, these genetically modified iAs are capable of being implanted in the central nervous system (for example, intracerebrally, or into spinal cord) or peripheral nervous system, and reproducing structural and/or functional characteristics of the disease or disorder.

### BRIEF DESCRIPTION OF DRAWINGS

FIG. 1. Molecular and functional characterization of iAs-B and iAs-SB. 1A, Experimental design for generating iAs from hESCs. Ubiq, ubiquitin; rtTA, reverse tetracycline-controlled transactivator; Puro, puromycin; hygro, hygromycin; EM, expansion medium; FGF, medium containing fibroblast growth factor; DM, maturation medium. 1B, Representative immunohistochemistry images (top) and quantification (bottom) of expression of GFAP, S100B, and VIM in iAs at 7, 10, 14, or 21 d after induction. 1C, The percentage of hESCs positive for the indicated markers after 7 d in culture relative to the starting amount. 1D, Ki67 labeling in iAs-B and iAs-SB at different time points after induction. 1E, Representative images showing immunolabeling of astrocyte specific markers and phalloidin at induction day 21 in iAs-SB. 1F, Representative high-magnification image of iAs-SB labeled for GFAP and phalloidin. 1E, 1F, n=3 independent experiments, with similar results. 1G, Glutamate uptake capacity of iAs-B and SB at day 14 and human primary adult astrocytes in culture. 1H, Bright-field image of an iAs-SB cell, 14 d after induction, being filled with biocytin (left, arrow), and the spreading of biocytin to neighboring cells (right) (representative of n=3 independent experiments with similar results). P, pipette. 1I, Fluo-4-based calcium-imaging results showing the percentage of cells that responded to ATP stimulation. 1J, Fold increase in IL-6 and CXCL10 expression after 8 h of treatment with 10 ng/ml IL-1β (cyt.) for both iAs-B and iAs-SB at days 14 and 21 after induction. Fold increase is normalized to levels of GAPDH and relative to levels in the control. *P<0.05, **P<0.01, ***P<0.001, ****P<0.0001, one-tailed ratio paired t-test comparing iAs with and without treatment. 1K, 1L, The presence of spontaneous postsynaptic currents (sPSCs) in induced neurons (iNs) that were co-cultured with primary mouse astrocytes (mAs; black; n=13 iNs) (1K), with iAs-SB (blue; n=6 iNs) (1K, 1L), or without glia (gray; n=6 iNs) (1L) for 32-35 d. Bar graphs show mean frequency (±SEM) of sPSCs. *P<0.05, unpaired two-tailed Mann-Whitney test. 1M, MAP2+ area covered by Bassoon- and PSD95-costaining (n=2 independent experiments with n=3 (mAs) or n=4 (no glia, B, and SB) biological replicates). *P<0.05, **P<0.01, one-way ANOVA comparing all groups, with Tukey correction for multiple comparisons. Normal distribution was assessed with a Shapiro—Wilk test. For immunohistochemistry images, Hoechst was used to stain nuclei. Data are shown as the mean ±SEM from n=3 (1B-D, 1G, 1I,), n=4 (1J), or n=2 (1M) independent experiments. Scale bars, 100 μm (1B, 1E), 10 μm (1F), or 25 μm (1H).

FIG. 2. Integration of iAs into mouse brain, and modeling of Alexander disease with CRISPR and iAs. 2A, Ratio of engrafted to total transplanted cells. 2C, 2B, Representative images of transplanted iAs positive for both GFP and VIM (2C) and ratio of GFP+VIM+cells to total GFP+cells (n=8 neonatal and 4 adult mice). 2D, Co-labeling of GFP+cells with the astrocytic protein AQP4 and the endothelial-specific protein CD31 in an overview (left), magnified view with orthogonal reconstruction (middle images), and 3D rendering (right). Similar results were observed with four neonatal and three adult transplanted mice. 2E, GFAP and S100B labeling of iAs-SB at 28 d after induction for WT and AxD cells. The arrow marks cells with GFAP inclusions. Similar results were observed in two independent experiments. 2F, ATP1B2 expression in AxD cells, normalized to GAPDH and relative to that in WT cells. *P<0.05, unpaired two-tailed t-test with Welch's correction for unequal variances. 2G, Fluo-4-based calcium-imaging results showing the fold increase in fluorescence in WT iAs and AxD iAs over time, before and after ATP addition (left), and the fold increase at the peak of the response (right). Results shown for 63 WT and 51 AxD responding cells. *P<0.05, multiple t-test comparing WT iAs to AxD iAs at each time point. Scale bars, 10 μm (2C, 2D) or 50 μm (e). Data are shown as the mean ±SEM. for n=8 (2A, 2B, pups) or 4 (2A, 2B, adults) mice, or for n=3 independent experiments (2F, 2G).

FIG. 3. Screening of transcription factors for astrocyte induction. 3A, Experimental design of screening experiments in hESCs. 3B Representative GFP-fluorescence images for different combinations of Sox9, Nfia and Nfib at 21 days of induction using GFAP::GFP lentivirus. Experiments were performed three independent times showing similar results. 3C, Representative GFAP::EGFP-fluorescence images of iAs-B and iAs-SB at 7, 14 and 21 days after induction and representative bright field images at 21 days. Boxes show zoom-in on cells indicated by arrows. Experiments were performed at least three independent times showing similar results. 3D, Representative phalloidin labeled cells for all factor combinations at 21 days of induction. Experiments were performed three independent times showing similar results. 3E, Representative images of S100B labeled cells for all factor combinations at 21 days of induction. Experiments were performed three independent times showing similar results. B=Nfib, SB=Sox9+Nfib, d=days. Scale bar=100 μm.

FIG. 4. Screening of factors to induce astrocytes by RT-qPCR and GFAP expression during the first week of induction. 4A, RT-qPCR showing expression of GFAP, S100B and ALDH1L1 after 21 days of induction for all combinations of Nfia, Nfib and Sox9. Results are the mean±SEM of three independent experiments (GFAP) or the mean of two independent experiments (S100B and ALDH1L1) and are presented as relative expression of the specific gene normalized to GAPDH expression. 4B, Quantification of GFAP protein expression by immunolabeling at indicated time points during the first week of induction. Percentage of cells positive for GFAP is shown as mean±SEM of three independent experiments.

FIG. 5. iAs-B induction and FACS analysis of iAs-B and iAs-SB populations. 5A, Representative images of iAs-B immunolabeled for indicated astrocytic markers. Experiments were performed three independent times showing similar results. 5B, Gating strategy used for FACS analyses. Left: forward vs side scatter. Middle: Singlets selection. Right: Viable cells based on 7-AAD staining. 5C, Contour plots showing CD44 expression. 5D, Contour plots showing VIM expression. Results are shown as mean±SEM from three independent experiments. 7 d=7 days, 21 d =21 days. Scale bar=100 μm.

FIG. 6. iAs morphology, proliferation and glycogen granules. 6A, Representative images of Ki67 staining for iAs-B and iAs-SB with positive cells marked with an arrow. Experiments were performed three independent times showing similar results. 6B, Representative immunolabeling of specific astrocytic markers as well as phalloidin at 21 days of induction in iAs-B. Experiments were performed three independent times showing similar results. 6C, Representative images of glycogen granules staining of iAs-B, SB, human primary astrocytes (hAs) and mouse astrocytes (mAs) and quantification of iAs displaying glycogen granules. Results are shown as mean±SEM from three independent experiments. 6D, Representative images showing immunolabeling for indicated astrocytic markers as well as phalloidin at 2 months after induction. Experiments were performed three independent times showing similar results. Scale bar=25 μm (6A), 100 μm (6B, 6d), 10 μm (6C).

FIG. 7. Induction of reprogramming factors and Dox dependence. 7A, Expression of human (endogenous) and mouse (exogenous) Nfib and Sox9 at days 0, 7, 14 and 21 with Dox-withdrawal at 7 or 14 days. Results are the mean±SEM of three independent experiments and presented as relative expression normalized to GAPDH. 7B, Representative images of iAs cultures labeled for GFAP and VIM after 21 days of induction with Dox withdrawal at 7 or 14 days. Experiments were performed three independent times showing similar results. 7C, Quantification of percentage positive cells for GFAP, S100B and VIM at 21 days of induction with Dox withdrawal at 7 or 14 days. Results are mean±SEM of three independent experiments and presented as percentage of positive cells normalized to the percentage without Dox withdrawal. An ordinary one-way ANOVA with Tukey test correction for multiple comparisons was used to detect significant differences. *P value<0.05. Scale bar =100 μm.

FIG. 8. Characterization of iAs derived from H9 hESC and human induced pluripotent stem cells (hiPSC) lines. Representative images showing immunolabeling for the astrocytic markers GFAP, S100B, GLAST and ALDH1L1 as well as phalloidin at 21 days of induction in iAs-B and iAs-SB. Experiments were performed three independent times showing similar results. Scale bar=100 μm.

FIG. 9. Real-time qPCR analysis of iAs. Comparison of expression of different astrocytic genes between iAs-B and iAs-SB at different time points and human adult and embryonic astrocytes. 9A, ALDH1L1, ALDOC, GFAP, GLAST, GLUL, HEPACAM. 9B, AQP4, GLT1, S100B. Results are the mean±SEM of three independent experiments and are presented as relative expression of the specific gene normalized to GAPDH expression. 9C, Single-cell quantitative RT-qPCR analysis (Fluidigm) of the expression of (top) house-keeping and induction, (middle) astrocytic, (bottom) stem cell, oligodendrocytic and neuronal genes. Expression levels are presented in Ct values and color-coded as shown on the top left. Lower Ct values (grey) correspond to higher mRNA expression levels. mRNA levels were quantified in single cells FACS-sorted by viability.

FIG. 10. Functional characterization of iAs. 10A, Representative images showing biocytin spreading in iAs-B at 21 days (left image) and iAs-SB at 21 days (right image). Experiments were performed three independent times showing similar results. 10B, Representative images showing Fluo-4 fluorescence in iAs-SB 2 seconds (2″) before adding ATP, response 20 seconds (20″) after ATP addition and recovery to basal levels 60 seconds (60″) after ATP stimulation and Fluo-4-based calcium imaging results showing the percentage of cells displaying spontaneous increases in fluorescence in iAs-B, SB and human primary adult astrocyte cultures. Results are the mean±SEM of three independent experiments. 10C, Fold increase in expression of CCL5 and CXCL8 after 8 hours of treatment with 10 ng/ml IL-1β for both iAs-B and SB at day 14 and 21 after induction. Results are the mean±SEM of four independent experiments and are presented as fold increase in expression normalized by GAPDH and relative to the control. Statistical analysis consisted in a one-tailed ratio paired t-test comparing iAs with and without treatment. cyt=IL−1β. *P value<0.05. **P value<0.01. ***P value<0.001. ****P value<0.0001. 10D, Presence of glutamatergic sPSCs is illustrated. Current traces show glutamatergic sPSCs recorded from iNs with mAs (left, black) or with iAs-SB (left, blue) in the presence of 100 μM Ptx and blocked in the presence of 50 μM D-APV +5 μM NBQX. Expanded current traces illustrate single glutamatergic sPSCs. Bar graph (right) show the frequency of sPSCs in the presence of Ptx and Ptx+D-APV+NBQX in iNs co-cultured with mAs (n=13 iNs) or with SB-iAs (n=6 iNs). An unpaired two-tailed Mann-Whitney test was used for statistical analysis. *P value<0.5 10E, Averaged glutamatergic sPSC detected from recordings in iNs co-cultured with mAs (black, n=39 sPSCs) or iAs-SB (blue, n=60 sPSCs) (top). Amplitude of averaged glutamatergic sPSCs from 6 (SB) or 13 (mAs) iNs are illustrated in the bar graph (bottom, p=0.7813). 10F, Pie chart (left) illustrating the amount of iNs with burst-like activity when co-cultured with mAs (black), iAs- SB (blue) or without glia (grey). Current traces from iNs co-cultured with mAs or iAs-SB containing burst-like events and the lack of burst-like events in current traces from iNs without glia (right). 10G, Representative images of co-localization analyses for PSD95, Bassoon and MAP2 staining of iNs without glia (No glia) or in co-culture with iAs-B, iAs-SB or mAs. Experiment was performed two independent times with 3 (mAs) or 4 (No glia, B and SB) biological replicates Scale bar=25 μm 10A, 10 μm 10B, and 5 μm 10C.

FIG. 11. Characterization of iAs transplanted into mouse brains. 11A, Experimental design for transplantation of eight neonatal and four adult mice. 11B, Representative image showing iAs produced in parallel to those used for transplantation and kept in culture until the day when mice were sacrificed. Experiments were performed three independent times showing similar results. 11C, Representative images of grafts of transplanted iAs labeled for human specific nuclear antigen (HuNu) in neonatal or adult mouse cortex and striatum at 4-5 weeks. Experiments were performed in four neonatal and four adult transplanted mice showing similar results. 11D, Representative image showing GFP+iAs co-labeled with VIM in adult transplanted brains. Experiments were performed in four neonatal and four adult transplanted mice showing similar results. 11E, Representative images of GFP+iAs co-labeled with S100B in neonatal (upper image) and adult (lower image) transplanted mice. Experiments were performed in four neonatal and four adult transplanted mice showing similar results. 11F, Representative orthogonal projections of GFP+ iAs co-labeled with GLAST in neonatal (upper image) and adult (lower image) transplanted mice. Experiments were performed in six neonatal and four adult transplanted mice showing similar results. 11G, Representative images of GFP+ cells transplanted in neonatal (upper image) or adult (lower image) mice in close interaction with Bassoon+/PSD95+ synaptic puncta. Experiments were performed in four neonatal and three adult transplanted mice showing similar results. 11H, Co-labelling of a GFP positive cell with antibodies for specific astrocytic protein AQP4 and for endothelial specific protein CD31 in an adult transplanted mouse. Experiments were performed in four neonatal and four adult transplanted mice showing similar results. 11I and 11J, Representative images of Cx43 staining in GFP labeled cells (left images) with orthogonal reconstruction (right images) in a neonatal and adult transplanted mouse. Experiments were performed in six neonatal and four adult transplanted mice showing similar results. 11K and 11L, Co-labelling of GFP positive transplanted cells filled with biocytin 6 weeks after transplantation (11K) and spreading to GFP+/HuNu+ transplanted as well as GFP−/HuNu− host cells 13 weeks after transplantation (11L). In each image, 2nd arrow from top marks injected cells, lower arrows mark transplanted cells, and top arrow mark host cells, with spreading of biocytin. Experiments were performed four independent times showing similar results. Scale bar=100 μm 11B, 200 μm 11C, 10 μm 11D, 11E, 11G, 11H, 5 μm 11F, 11I, 11J, and 20 μm 11K, 11L. LV=lateral ventricle.

FIG. 12. Genome-engineering strategy for AxD, absence of off-target editing and characterization of AxD-iAs. 12A, Vector construct (left) encoding Cas9n and sgRNAs 1 and 2 targeting regions near the R239C mutation site in exon 4 of GFAP (underlined). Repair template (ssDNA) that introduced the AxD mutation (in red) and a silent change in one PAM sequence (in green) to avoid repetitive editing (middle). Sequencing results of one homozygous clone obtained showing both changes in the genome (right). 12B, Expression of S100B at 21 days of differentiation for WT- and AxD-iAs. Results are the mean±SEM of three independent experiments and are presented as relative expression of the specific gene normalized by GAPDH expression. 12C, GFAP and S100B labeling of iAs-SB at 28 days after induction for the WT and the AxD lines. Arrows mark cells with GFAP inclusions. Experiments were performed two independent times showing similar results. 12D, Expression of KCNJ10 at 14 and 21 days of differentiation for WT- and AxD-iAs. Results are the mean±SEM of three independent experiments and are presented as relative expression normalized by GAPDH expression. An unpaired two-tailed t-test with Welch's correction for unequal variances was performed. *P value<0.05. 12E, Percentage of cells responding to ATP in calcium imaging assays for WT- and AxD-iAs at 21 days of differentiation. Results are the mean±SEM of three independent experiments. 12F, Fluo-4-based calcium imaging results showing the fold increase at the peak of the response (right). Results are the mean±SEM of 63 (WT) and 51 (AxD) responding cells analyzed from three independent experiments. Box-whisker plot shows from down to top, the minimum, first quartile, median, third quartile and maximum values. An unpaired two-tailed Mann-Whitney test was used to detect significant differences. *P value<0.05. Scale bar=50 μm.

### DESCRIPTION

Herein disclosed is a controlled and simple protocol to rapidly and efficiently induce highly enriched, mature, and bona fide functional human astrocytes. By “functional” herein is meant the ability to display previously described astrocytic functional properties at similar efficiency as compared to normal human or mouse astrocytes in vitro. Examples of functional astrocyte include, but are not limited to, (1) the expression of glutamate transporters such as GLT1 and GLAST and the ability to take up glutamate added to the culture medium; (2) the expression of gap junction proteins such as Cx43, establishment of gap junctions between cells and the ability to transfer biocytin between cells trough gap junctions; (3) the ability to show spontaneous and induced elevations of intracellular calcium levels; and (4) the ability to increase expression of cytokines upon an inflammatory stimulus such as exposure to IL-1beta; and (5) the ability to support formation of synapses in co-cultured neurons, or combination thereof

By overexpressing Nfib and/or Nfia alone or in combination with Sox9 in hESCs or hiPSCs, it has been possible to generate astrocytes that closely resemble adult human primary astrocytes at the phenotypic, molecular, and functional levels. These iAs are useful for studies on astrocyte biology and for neurological disease modeling.

Overexpression of the transcriptions factor(s), Nfib, Nfia, or Nfib and Sox9, or Nfia and Sox9 is all that is needed to induce astrocyte differentiation, in a period of 7-14 days. By “overexpression” herein is meant a higher level of expression compared to that in human pluripotent stem cells not bearing an expression vector for the transcription factor, which would be the baseline level of expression. Thus exogenous expression of these transcription factors should be under the control of a switchable promoter system, especially one that is inactive under baseline conditions and active in a manipulated condition, typically the presence of an added reagent. Flexibility of experimental design for use of the iAs is enhanced by use of a manipulated condition that can be easily avoided. In the working examples below the switchable promoter system is the reverse tetracycline-controlled transactivator and the manipulated condition is the inclusion of doxycycline (Dox) in the culture medium, however, while this is a robust and well-understood expression control system, others are known in the art, and any other expression control system with similar attributes would be similarly appropriate. Use of the reverse tetracycline-controlled transactivator system in particular is not essential, though it can be preferred in some embodiments.

As exogenous overexpression of the transcription factors is a temporary requirement, which need not be maintained (and in some embodiments, is preferably not maintained) after the pluripotent stem cells have differentiated into astrocytes, either integrating or episomal expression vectors are used . Alternatively, RNA (e.g. self-replicating RNA), or transgene expression from a safe harbor locus is used. The expression vector will typically encode a selectable marker, for example, an antibiotic resistance, in addition to the transcription factor. In the working examples below a lentiviral expression vector system is used. While this is a robust and well-understood expression vector system, many other expression vectors and their properties are known in the art, and any other expression vector system operable in the cells in the astrocyte differentiation pathway would be similarly appropriate. Use of a lentiviral expression vector in particular is not essential, though it can be preferred in some embodiments.

Some astrocytic phenotypes are stably established within seven days overexpression of Nfib or Nfia alone, Nfib plus Sox9, Nfia plus Sox9, or Nfib plus Nfia plus Sox9 (for example, expression of S100B or VIM), whereas the number of cells displaying the phenotype will diminish somewhat unless overexpression is maintained for a longer period of time, up to 14 days (for example, expression of GFAP). Thus, iAs become independent of the exogenous overexpression the transcription factors between 7 and 14 days of such treatment. In some embodiments the lower yield of fully differentiated iAs is acceptable and overexpression of the transcription factors is not maintained past 7 days. In other embodiments, overexpression of the transcription factors is maintained for 8, 9, 10, 11, 12, 13, or 14 days, for example, because a better yield of fully differentiated iAs is desired.

A detailed exemplary protocol is described in the examples below, but in general human pluripotent stem cells (either embryonic stem cells “ESCs”, or induced pluripotent stem cells “iPSCs”) are dissociated (for example, with a protease preparation) and plated on an appropriate surface (e.g., a MATRIGEL-coated surface) and cultured in an appropriate medium for a day. The cultured pluripotent stem cells are then exposed to the expression vector and cultured to allow incorporation of the vector. After sufficient time (for example, overnight culture) for the vector to be incorporated (integrated, in the case of an integrating vector) the culture conditions are manipulated to switch on expression from the vector so that the Nfib, Nfia, Nfib+ Nfia, Nfib+Sox9, Nfia+Sox9, or Nfib+Nfia+Sox9, and the selectable markers are expressed. After an interval of time for expression to become established (for example, overnight culture) the culture medium is changed to an Expansion Medium supplemented with the selection reagent(s) for the selectable marker carried by the expression vector and the factor required to keep the expression system switched on (expression factor). This allows the cells to recover from infection with the viral vector and from selection, and also allows for a degree of proliferation. Over the course of several days, with daily medium changes, a mixed medium containing decreasing amounts of Expansion Medium and increasing amounts of an astrocyte-appropriate (alternatively termed astrocyte-permissive) medium (for example, FGF Medium) is provided to the culture, until the medium is 100% the astrocyte-appropriate medium at which point inclusion of the selection reagents is discontinued; the expression factor is included throughout. FGF medium promotes the expression of glutamate transporters and thus induces glutamate uptake capabilities. Over this period of time, cells that did not incorporate the expression vector, or from which it has been lost, have been eliminated by the selection reagent, and expression of the transcription factors promote differentiation. The day after reaching 100% astrocyte-appropriate medium, the cells are dissociated, pelleted, and replated on an appropriate surface and cultured for a few days in the astrocyte-appropriate medium supplemented with the expression factor. Subsequently, half of the culture medium is replaced with a Maturation Medium every 2-3 days. During this interval the cells take on a more mature morphology and develop a more mature phenotype. The Maturation Medium can be supplemented with the expression factor, which is preferably present through the 14th day of culture. The expression factor can be supplied indefinitely, to ensure expression of the transcription factors is not lost, but can also be withdrawn temporarily or permanently to avoid interference with experimental measurements, for example if analyzing global gene expression. In some embodiments, the exposure to the expression vector takes place on day −1 of the procedure. In some embodiments, the expression factor is first included on day 0 of the procedure. In some embodiments, the selection reagents are first included on day 1 of the procedures. In some embodiments, use of mixtures of Expansion Medium and astrocyte-appropriate medium commences on day 3 of the procedure and 100% astrocyte-appropriate medium is reached on day 6 of the procedure. In some embodiments, dissociation and re-plating takes place on day 7 of the procedure. In some embodiments, replacement of half of the medium commences on day 10 of the procedure.

This method of producing iAs offers advantages in speed, simplicity (fewer steps, less complex and cheaper media) robustness, and characterization of the iAs produced, as compared to previous methods (see Table 1). A particular advantage is that there is no need to first perform a neural induction, nor a need to make embryoid bodies. Perhaps the most immediately recognizable advantage is the speed with which the iAs are generated. Important astrocyte biomarkers are observed by seven days for example, by flow cytometry or immunohistochemistry, and as early as by three days by QPCR, after exogenous expression of the transcription factor(s) commence. This compares with from 4 to 84 weeks for biomarker appearance in earlier protocols. Moreover the portion of the population of cells that are positive for the biomarker is at least similar to, if not greater than, that observed in the earlier protocols. Various functional characteristics of astrocytes are observed in the iAs produced by the herein disclosed protocol within 14-21 days. In comparison, the earlier protocols required from 4 to 71 weeks for these various functional characteristics to be observed, if the cells were even assessed for their presence. Table 1 includes seven functional characteristics of astrocytes for which the iAs produced by the herein disclosed protocol have been characterized; none of the iAs produced by the earlier protocol have been characterized for more than 4 of these functional characteristics. This more complete characterization means that iAs produced by the herein disclosed protocol can be used with greater confidence that they faithfully represent natural astrocytes, and provides a larger number of assayable features for whatever use may be made of them.

TABLE 1
Comparison of methods to generate astrocytes from human pluripotent stem cells.

| | | | | | | | | | | | | | | | | | | Transplantation |
|---|---|---|---|---|---|---|---|---|---|---|---|---|---|---|---|---|---|---|
| | | | | Astrocyte biomarkers | | | | Glycogen | Glutamate | | Calcium | | Synapse | Cytokine | | Functional | | differemtiation + |
| Protocol | Cells | Time | | S100B | GFAP | VIM | Others | granules | uptake | | imaging | | support | stimulation | | gap-junctions | | survival time |
| Present | hESC | 7 | days | >90% | >90% | >90% | ALDH1L1 | 21 days | 14 | days | 14 | days | 7 days + | 14 | days | 14 | days | 7-10 days + |
| disclosure | hiPSC | | | | | | GLAST | | | | | | 32-35 days | | | | | 4-13 weeks |
| | | | | | | | CD44 | | | | | | co-culture | | | | | |
| 1-3 | hESC | 6 | weeks | >95% | >95% | >95% | CD44 | | 6 | weeks | | | 6 weeks + | | | | | 6 weeks + |
| | hiPSC | | | | | | NFIA | | | | | | 14 days | | | | | 1-6 weeks |
| | | | | | | | | | | | | | ACM** | | | | | |
| 4 | hiPSC | 7-10 | weeks | >90% | >90% | >85% | GLAST | | 7-10 | weeks | 7-10 | weeks | 7-10 weeks + | | | | | |
| | | | | | | | NFIA | | | | | | 21 days | | | | | |
| | | | | | | | | | | | | | co-culture | | | | | |
| 5 | hESC | 13-26 | weeks | >90% | >90% | | ALDH1L1 | | 29 | weeks | 25 | weeks | 25 weeks + | | | | | 25 weeks + |
| | hiPSC | | | | | | CD44 | | | | | | 21 days | | | | | 4-14 weeks |
| | | | | | | | GLT1 | | | | | | co-culture | | | | | |
| | | | | | | | NFIA | | | | | | | | | | | |
| 6 | hESC | 11-13 | weeks | >95% | >70% | ? | ALDH1L1 | | 12-14 | weeks | 12-14 | weeks | | 13 | weeks | | | 11-13 weeks + |
| | hiPSC | | | | | | ALDOC | | | | | | | | | | | 2-7 weeks |
| | | | | | | | CD44 | | | | | | | | | | | |
| | | | | | | | CX43 | | | | | | | | | | | |
| | | | | | | | GLT1 | | | | | | | | | | | |
| | | | | | | | NFIA | | | | | | | | | | | |
| 7 | hESC | 7 | weeks | >90% | ? | | ALDH1L1 | | 9 | weeks | 8-10 | weeks | | 7 | weeks | 8 | weeks | |
| | hiPSC | | | | | | CD44 | | | | | | | | | | | |
| | | | | | | | GLAST | | | | | | | | | | | |
| | | | | | | | GLT1 | | | | | | | | | | | |
| | | | | | | | NFIA | | | | | | | | | | | |

| 8 | hiPSC | 14-84 | weeks | | ? | | HEPACAM | 22-60 | weeks | | | 25-71 weeks + | | | |
|---|---|---|---|---|---|---|---|---|---|---|---|---|---|---|---|
| | | | | | | | | | | | | 14 days | | | |
| | | | | | | | | | | | | co-culture | | | |
| 9 | hESC | 13 | weeks | ? | ? | ? | ALDH1L1 | | | 13 weeks | | | | | |
| | | | | | | | CX43 | | | | | | | | |
| | | | | | | | GLAST | | | | | | | | |
| | | | | | | | GLT1 | | | | | | | | |
| | | | | | | | GLUL | | | | | | | | |
| 10 | hESC | 13-19 | weeks | ? | ? | | CD44 | | | | | | 19 weeks | | |
| | hiPSC | | | | | | CX43 | | | | | | | | |
| | | | | | | | GLAST | | | | | | | | |
| | | | | | | | GLUL | | | | | | | | |
| | | | | | | | NFIA | | | | | | | | |
| 11 | hiPSC | 4 | weeks | >70% | >50% | | ALDH1L1 | | | | | | | | 20 weeks + |
| | | | | | | | AQP4 | | | | | | | | 1-3 weeks |
| | | | | | | | GLAST | | | | | | | | |
| 12 | hiPSC | 24 | weeks | >80% | >50% | | CD44 | | | | | 24 weeks + | | | |
| | | | | | | | | | | | | 14 days | | | |
| | | | | | | | | | | | | co-culture | | | |
| 13, 14 | hESC | 4 | weeks | >40% | 10-15% | ? | ALDH1L1 | | | 6 weeks | | | | | |
| | hiPSC | | | | | | GLT1 | | | | | | | | |
| 15, 16 | hESC | 5 | weeks | ? | 55-80% | | AQP4 | 5 | weeks | | | | | | 5 weeks + |
| | hiPSC | | | | | | GLAST | | | | | | | | 2 weeks |
| 17 | hESC | 9 | weeks | >90% | >95% | | AQP4 | 9 | weeks | | | | | | |
| | | | | | | | GLAST | | | | | | | | |
| 18 | hiPSC | 4 | weeks | >70% | >5% | >85% | CD44 | 4 | weeks | 4 | weeks | | 4 | weeks | |
| | | | | | | | FABP7 | | | | | | | | |
| | | | | | | | GLAST | | | | | | | | |
| | | | | | | | NFIA | | | | | | | | |
| | | | | | | | SOX9 | | | | | | | | |

Comparison of present disclosure with recent key protocols for differentiation of human pluripotent stem cells to astrocytes. Shown are time required and efficiency generating astrocytic protein expression, time to reach functionality and time points for transplantation. ? = not quantified. Blank box = not analyzed.
**ACM = astrocyte conditioned medium.
Table 1 References
The protocol of the present disclosure has also been published: Canals et al. Rapid and efficient induction of functional astrocytes from human pluripotent stem cells. Nat. Methods 15: 693-696 (2018), including online supplemental information, which is incorporated herein by reference in its entirety.
1. Jiang, P. et al. hESC-derived Olig2+ progenitors generate a subtype of astroglia with protective effects against ischaemic brain injury. Nat Commun 4: 2196 (2013).
2. Chen, C. Y. et al. Inhibition of Notch signaling facilitates the differentiation of human induced pluripotent stem cells into neural stem cells. Mol Cell Biochem 395: 291-298 (2014).
3. Jiang, P. et al. Human iPSC-Derived Immature Astroglia Promote Oligodendrogenesis by Increasing TIMP-1 Secretion. Cell Rep 15: 1303-1315 (2016).
4. Serio, A. et al. Astrocyte pathology and the absence of non-cell autonomy in an induced pluripotent stem cell model of TDP-43 proteinopathy. Proc. Natl. Acad. Sci. U S A 110: 4697-4702 (2013).
5. Krencik, R., Weick, J. P., Liu, Y., Zhang, Z. J. & Zhang, S. C. Specification of transplantable astroglial subtypes from human pluripotent stem cells. Nat Biotechnol 29: 528-534 (2011).
6. Roybon, L. et al. Human stem cell-derived spinal cord astrocytes with defined mature or reactive phenotypes. Cell Rep 4: 1035-1048 (2013).
7. Santos, R. et al. Differentiation of Inflammation-Responsive Astrocytes from Glial Progenitors Generated from Human Induced Pluripotent Stem Cells. Stem Cell Reports 8, 1757-1769 (2017).
8. Sloan, S. A. et al. Human Astrocyte Maturation Captured in 3D Cerebral Cortical Spheroids Derived from Pluripotent Stem Cells. Neuron 95: 779-790 e776 (2017).
9. Dezonne, R. S. et al. Derivation of Functional Human Astrocytes from Cerebral Organoids. Sci. Rep 7: 45091 (2017).
10. Holmgyist, S. et al. Generation of human pluripotent stem cell reporter lines for the isolation of and reporting an astrocytes generated from ventral midbrain and ventral spinal cord neural progenitors. Stem Cell Res 15: 203-220 (2015).
11. Sareen, D. et al. Human induced pluripotent stem cells are a novel source of neural progenitor cells (iNPCs) that migrate and integrate in the rodent spinal cord. J Comp Neurol 522: 2707-2728 (2014).
12. Krencik, R. et al. Dysregulation of astrocyte extracellular signaling in Costello syndrome. Sci Transl Med 7: 286ra266 (2015).
13. McGivern, J. V. et al. Spinal muscular atrophy astrocytes exhibit abnormal calcium regulation and reduced growth factor production. Glia 61: 1418-1428 (2013).
14. Patitucci, T. N. & Ebert, A. D. SMN deficiency does not induce oxidative stress in SMA iPSC-derived astrocytes or motor neurons. Hum Mol Genet 25: 514-523 (2016).
15. Emdad, L., D'Souza, S. L., Kothari, H. P., Qadeer, Z. A. & Germano, I. M. Efficient differentiation of human embryonic and induced pluripotent stem cells into functional astrocytes. Stem Cells Dev 21: 404-410 (2012).
16. Mormone, E., D'Sousa, S., Alexeeva, V., Bederson, M. M. & Germano, I. M. “Footprintfree” human induced pluripotent stem cell-derived astrocytes for in vivo cell-based therapy. Stem Cells Dev 23: 2626-2636 (2014).
17. Gupta, K. et al. Human embryonic stem cell derived astrocytes mediate non-cellautonomous neuroprotection through endogenous and drug-induced mechanisms. Cell Death Differ 19: 779-797 (2012).
18. Lundin, A. et al. Human iPS-Derived Astroglia from a Stable Neural Precursor State Show Improved Functionality Compared with Conventional Astrocytic Models. Stem Cell Reports 10: 1030-1045 (2018).
Foregoing references 1-18 are incorporated herein by reference for all that they teach about the differences between themselves and the presently disclosed protocol.

### EXAMPLES

The following non-limiting examples are provided for illustrative purposes only in order to facilitate a more complete understanding of representative embodiments now contemplated. These examples should not be construed to limit any of the embodiments described in the present specification. The inventor(s) published a version of these experiments as Canals et al., Nature Methods 15:693-696 (2018), which is incorporated by reference herein in its entirety.

Example 1

Glioqenic Transcription Factor Nfib Alone or Together with Sox9 Efficiently Induced Expression of Astrocyte Biomarkers

Glial fibrillary acidic protein (GFAP) is an intermediate filament protein expressed by astrocytes amongst other cell types and is considered the hallmark intermediate filament protein of astrocytes. GFAP is widely used as an immunohistochemical marker of astrocytes, though it is not universally detectable in all astrocytes and can be found in other cell types in the central nervous system and in cells of the extended astroglial family elsewhere in the body.

S100B calcium-binding protein B (S100B) is a glial specific protein expressed primarily by astrocytes, particularly mature and NG2-expressing astrocytes. Thus, S100B is also useful as a biomarker of astrocytes.

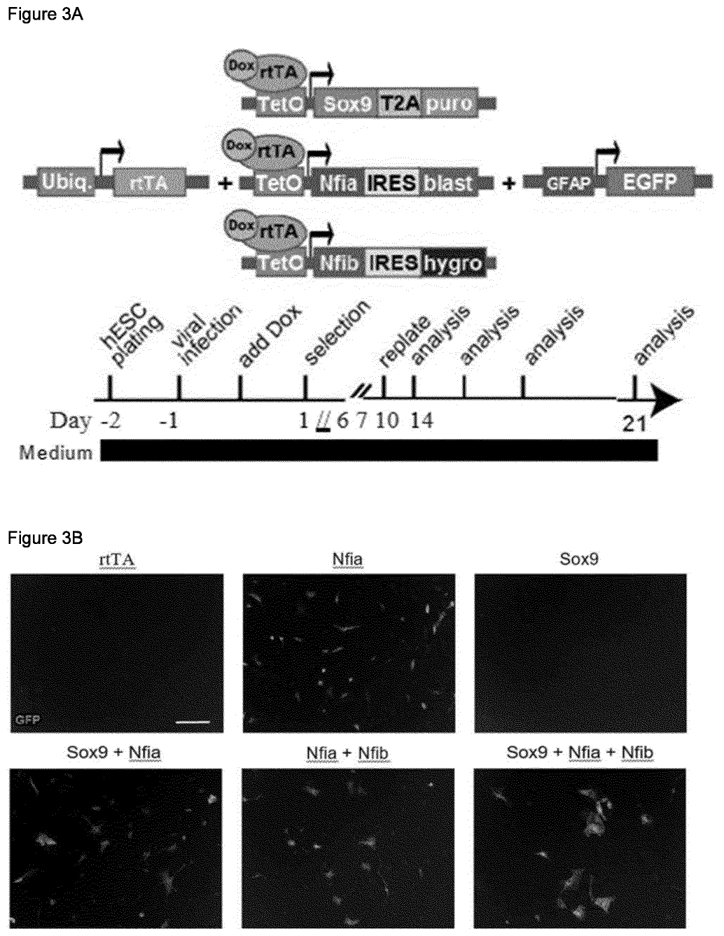

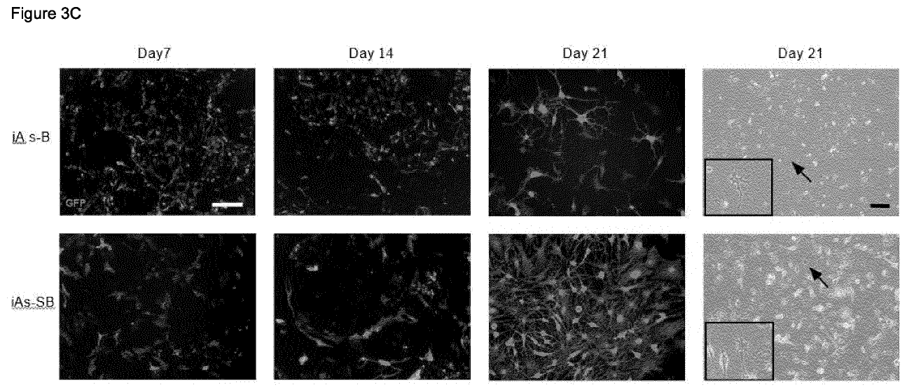

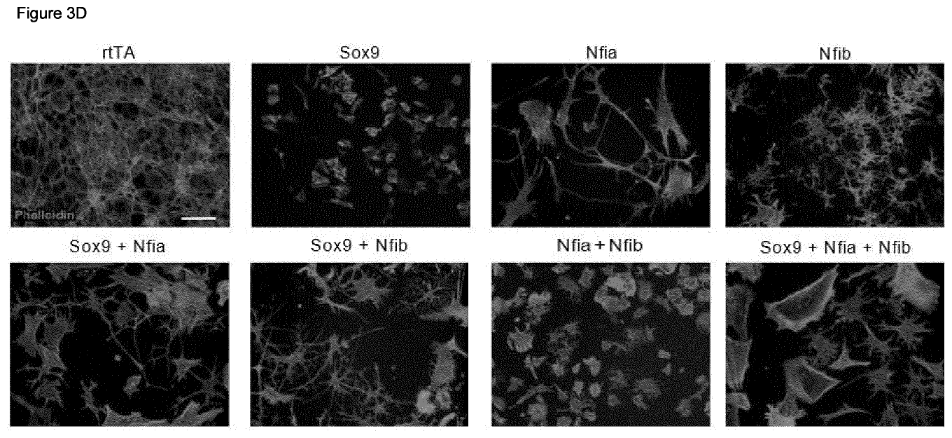

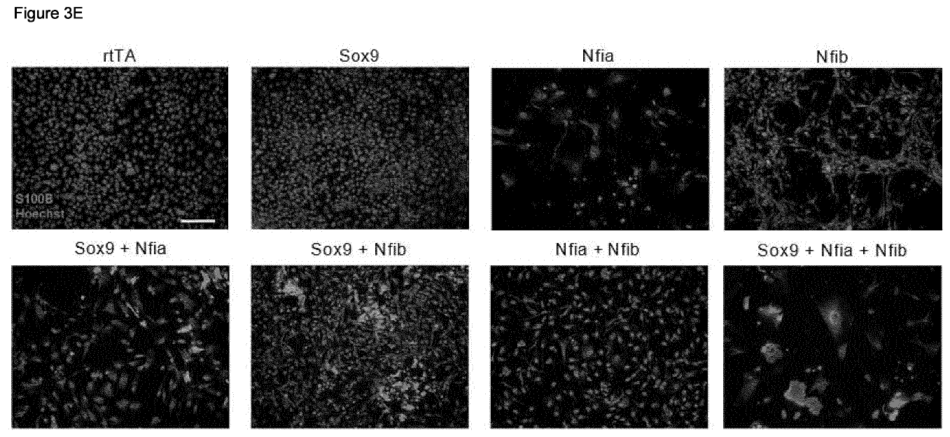

To test the ability of the gliogenic transcription factors Nfia, Nfib, and Sox9 to induce astrocytic phenotypes, human embryonic stem cells (hESC) where infected with a combination of lentiviral vectors capable of expressing these factors under control of a Tet-On (rtTA) expression system. The cells were also infected with a lentiviral vector expressing enhanced green fluorescent protein (eGFP) under control of the GFAP promoter (GFAP::eGFP) (FIG. 3A). Both Nfia and Nfib were able to induce eGFP expression through the GFAP promoter. (FIG. 3B).

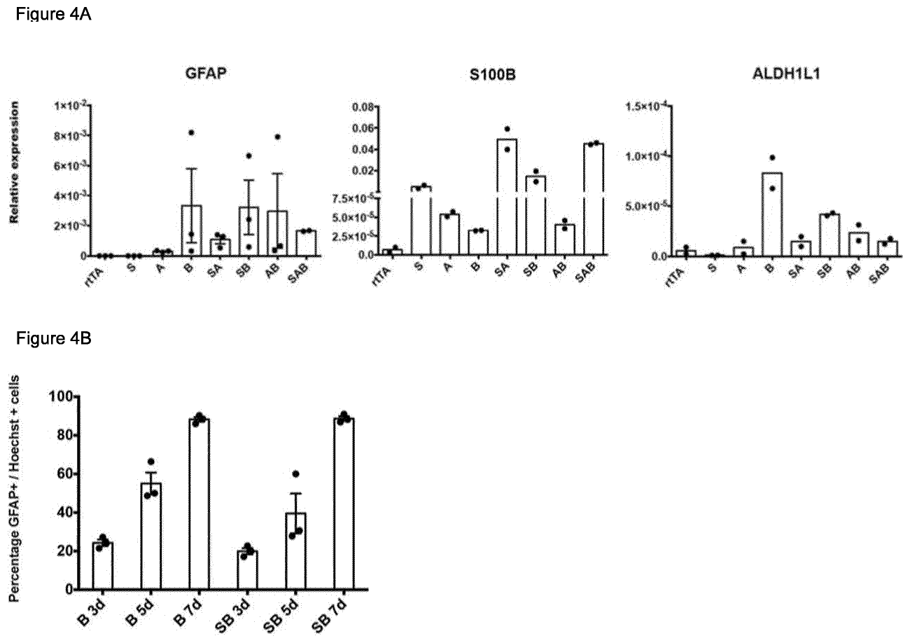

After screening different combinations of transcription factors, we found that Nfib alone (B) or together with Sox9 (SB) showed the most efficient induction of a co-transduced GFAP::EGFP reporter and S100B expression, with cells displaying stellate morphology, which is characteristic of astrocytes, after 14 days. Using RT-qPCR (reverse transcriptase-quantitative polymerase chain reaction), we found higher expression of GFAP and ALDH1L1 in cells that were co-transduced with combinations that included Nfib, whereas S100B expression was higher in cells treated with combinations that included Sox9 (FIG. 4A).

### Example 2

### Phenotypic Characterization of Induced Astrocytes

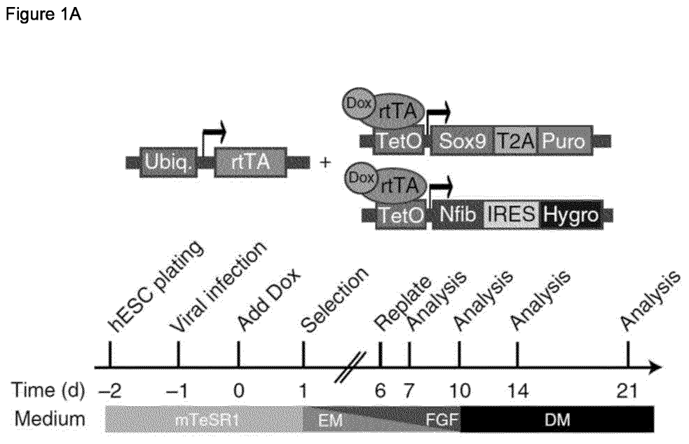

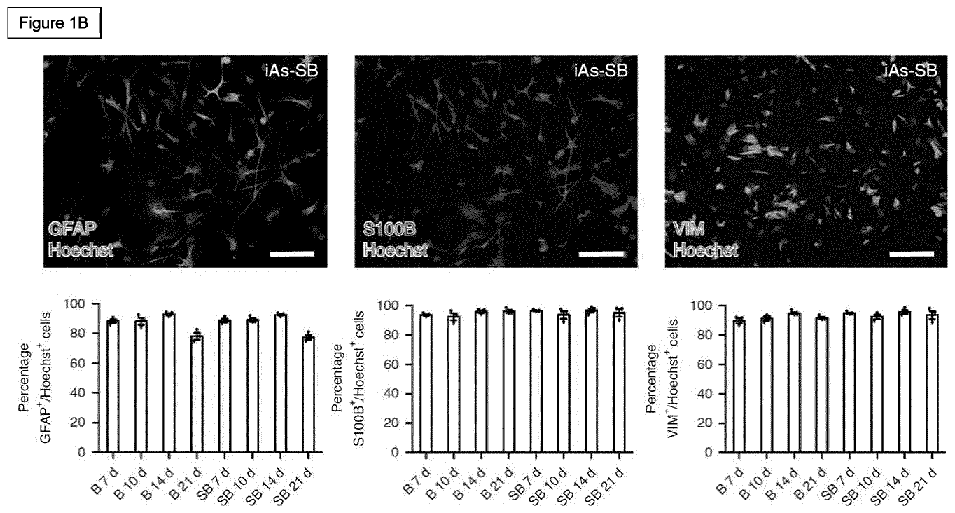

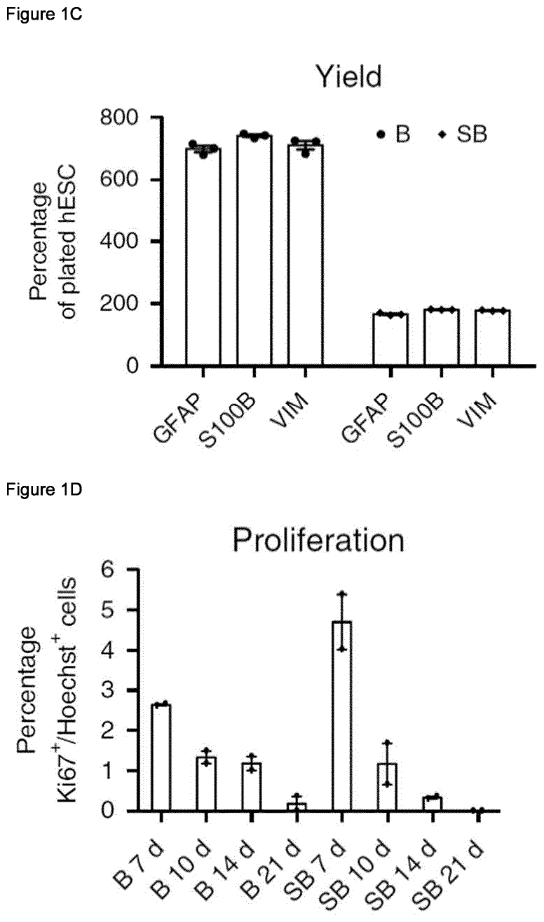

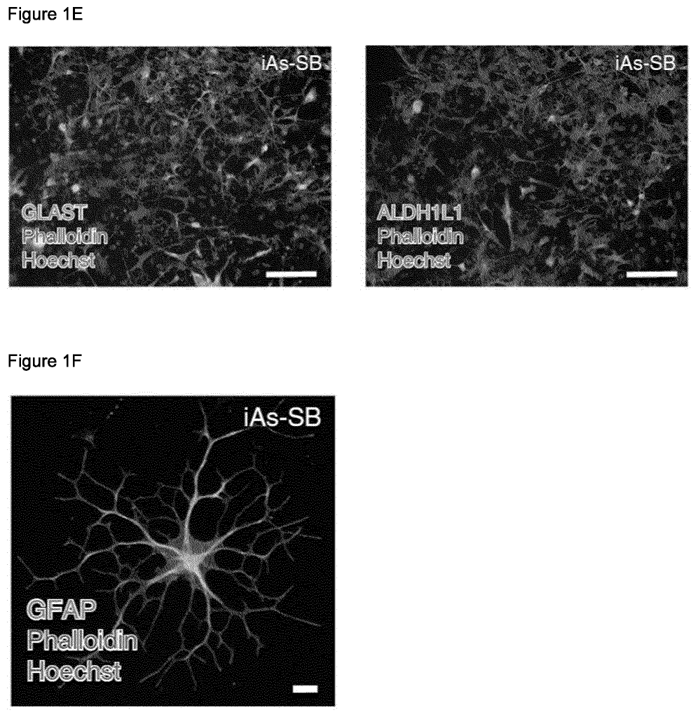

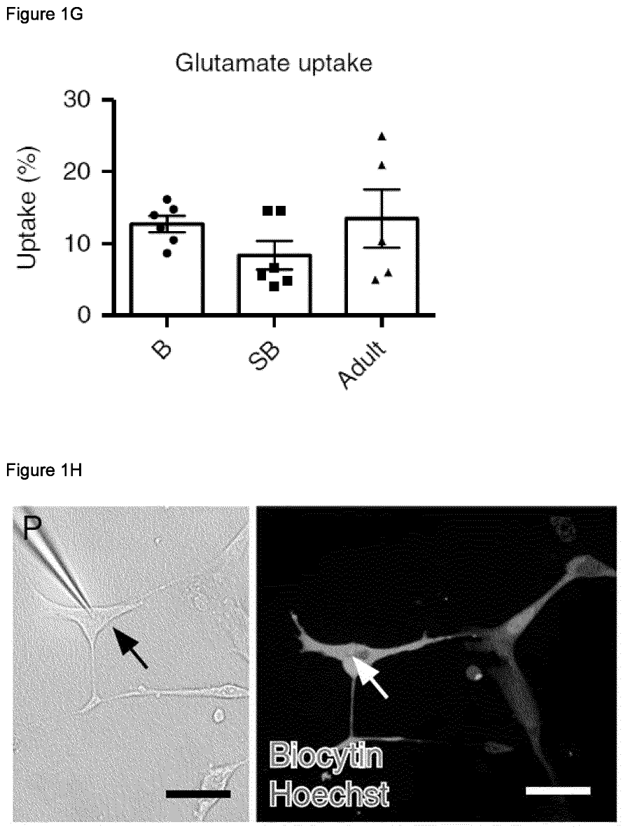

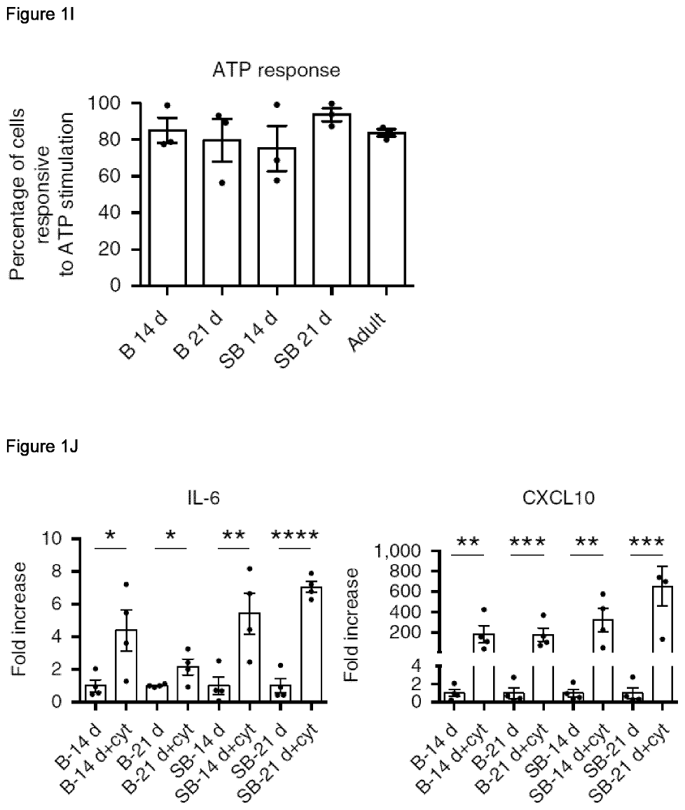

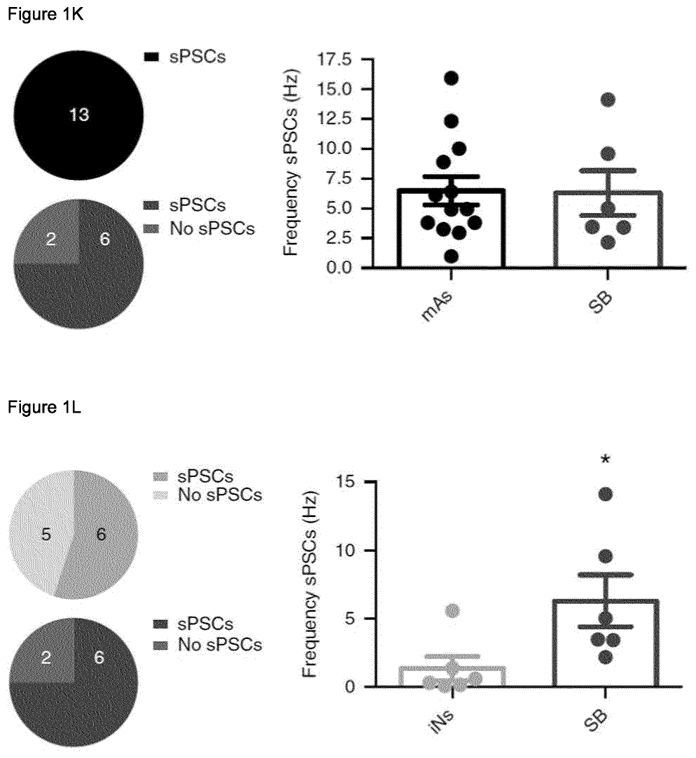

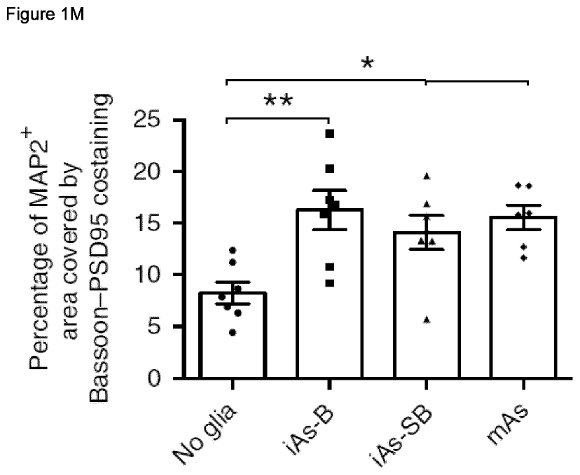

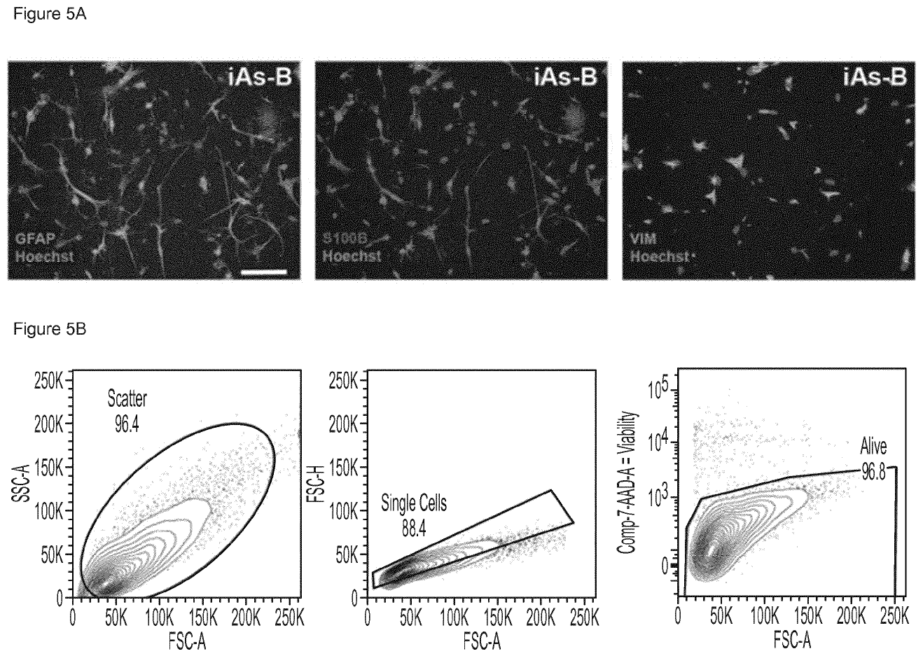

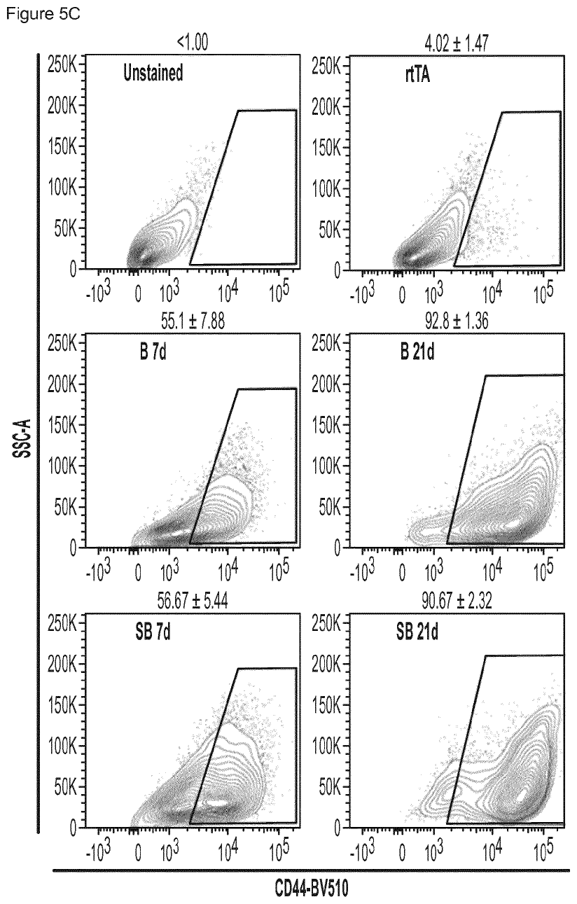

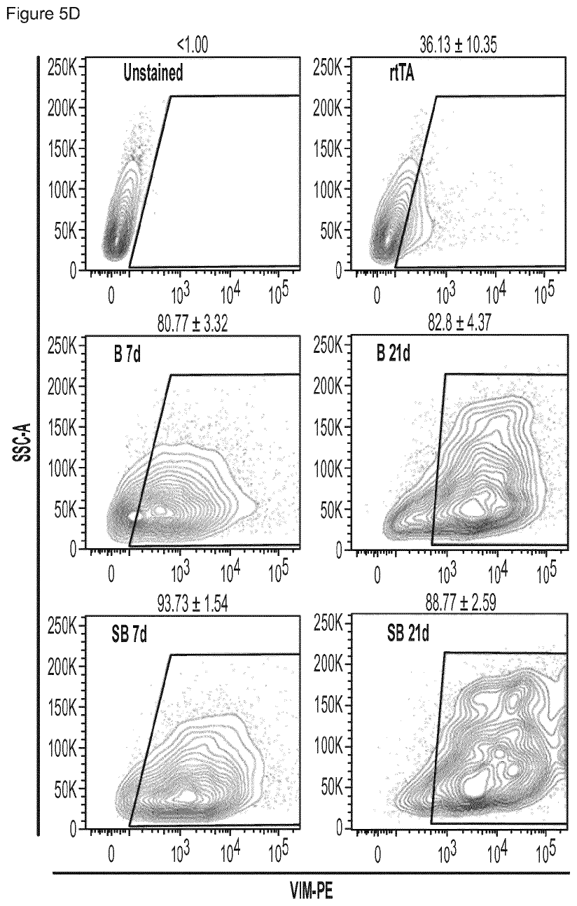

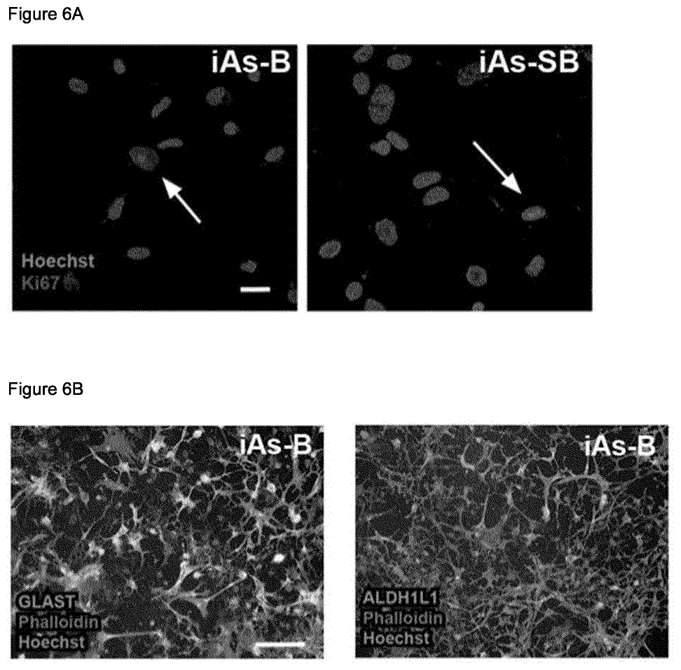

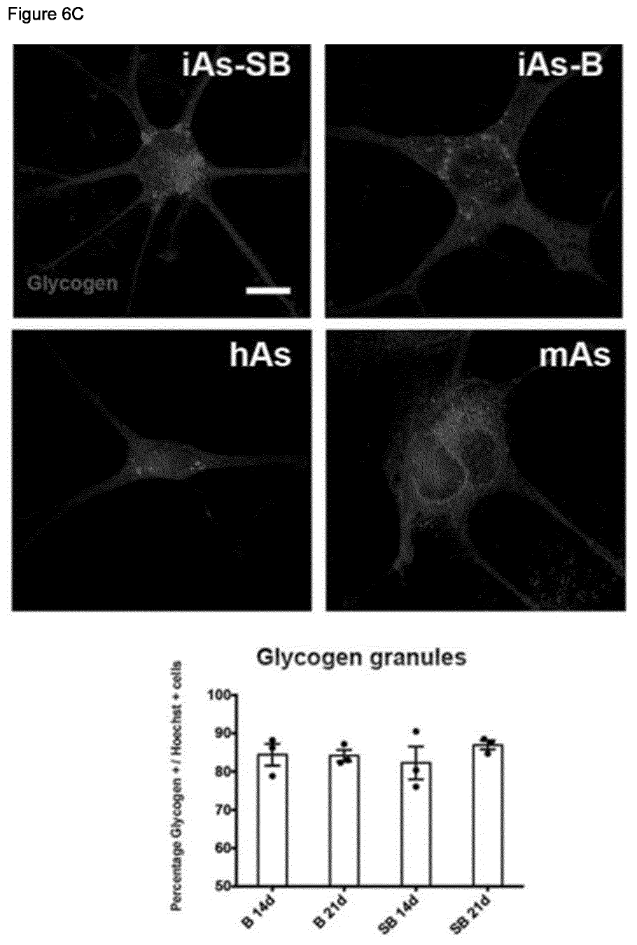

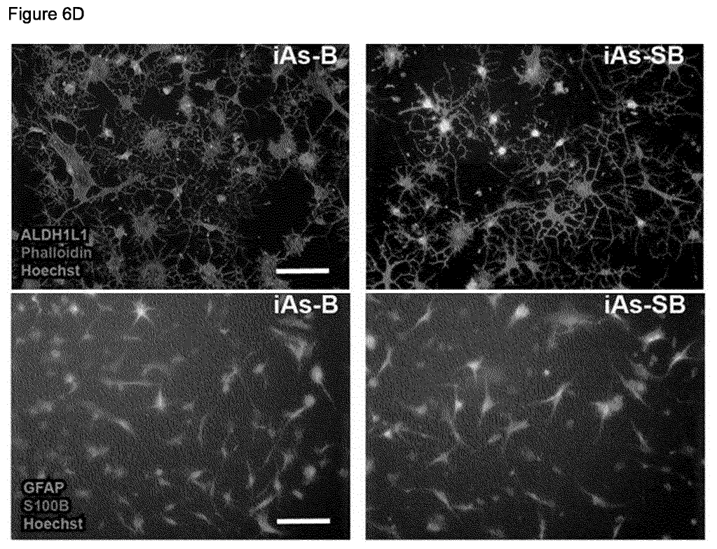

The induced astrocytes (iAs) that were obtained after treatment with Nfib (iAs-B) or with both Sox9 and Nfib (iAs-SB) (FIG. 1A) were further characterized. In both groups, GFAP expression was detected 3 days after induction, was observed to increase by day 5, and was found in a majority (−88%) of cells after only 7 days (FIG. 4B), together with S100B (iAs-B, 94%; iAs-SB, 96%) and vimentin (VIM; iAs-B, 90%; iAs-SB, 95%) expression (FIGS. 1C and 5A). Efficient induction of VIM and CD44 was also confirmed by FACS (FIG. 5B-D). The yield of GFAP⁺, S100B+, and VIM⁺ cells at 7 days was —700% (for B) and ˜180% (for SB) of the number of hESCs that had been plated (FIG. 1C). The expression of canonical astrocytic markers was stably maintained until 21 days, with the exception of GFAP, levels of which decreased slightly (FIG. 1B), consistent with the known upregulation of GFAP due to the presence of serum early in the protocol. Mature postnatal astrocytes are postmitotic, and at 21 days after induction, we found that proliferating (Ki67⁺) cells were extremely rare (iAs-B) or absent (iAs-SB) among iAs (FIGS. 1D and 6A). We also confirmed expression of ALDH1L1 and GLAST, as well as mature morphology with complex arborizations at day 21 after induction (FIGS. 1E, 1F, and 6B).

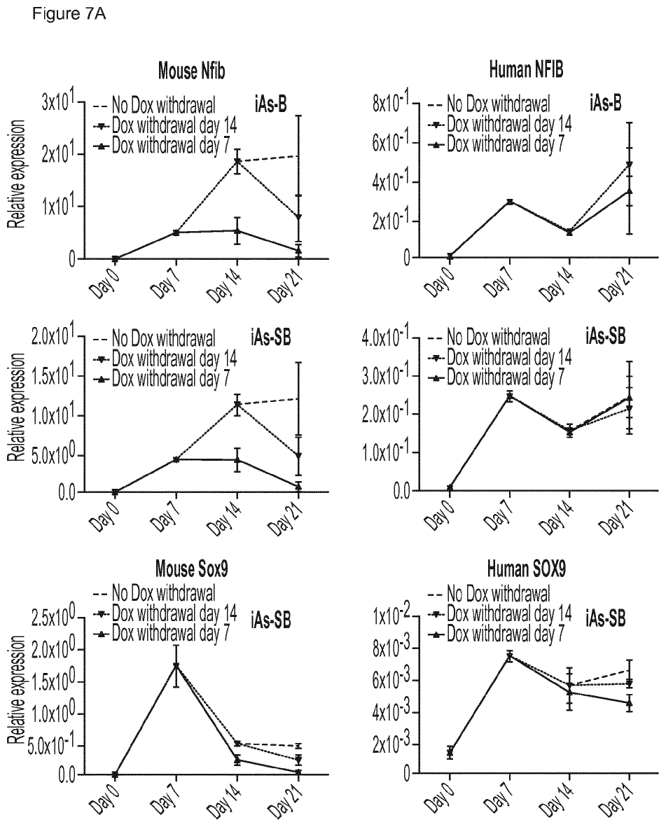

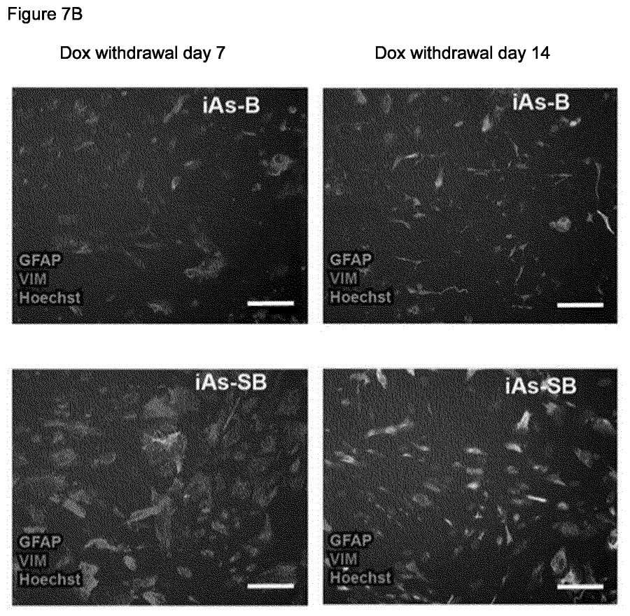

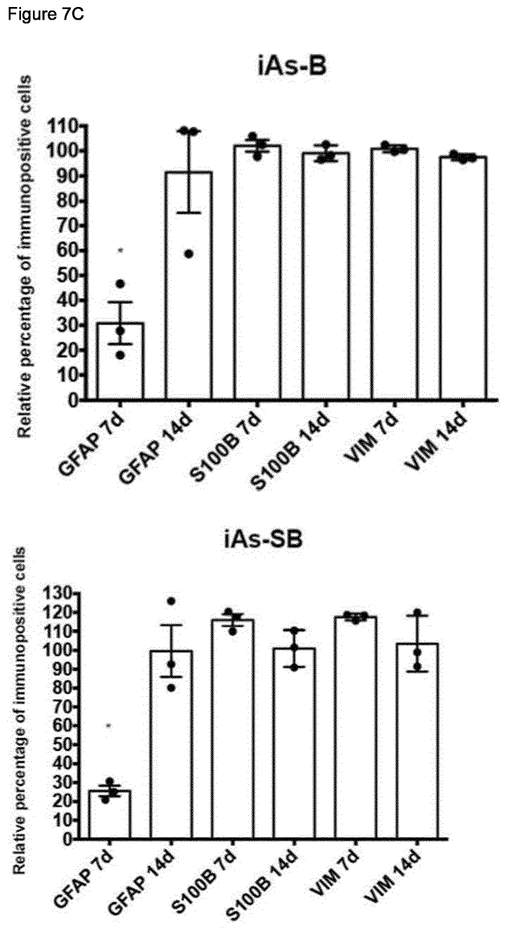

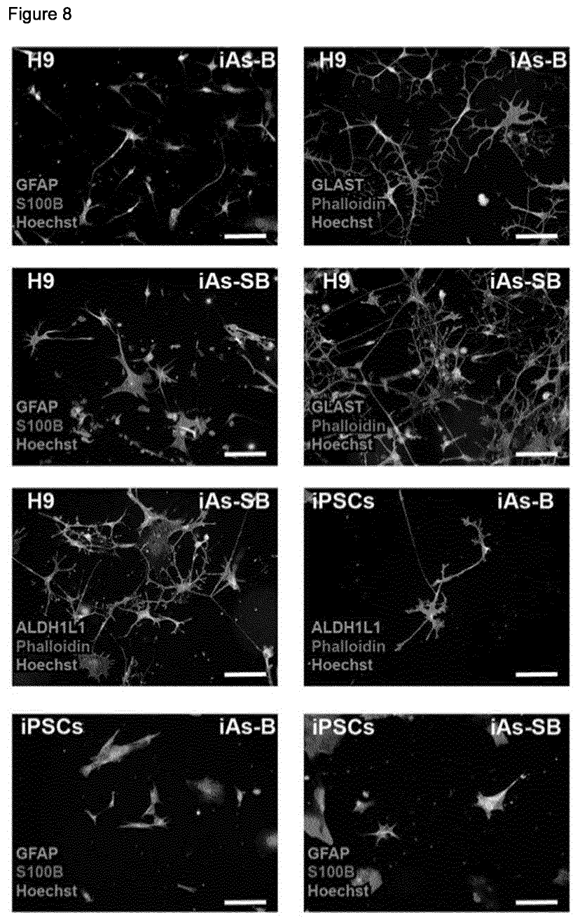

Astrocytes are the main storage sites of glycogen in the central nervous system. We detected glycogen granules in >80% of iAs, similar to what we observed in primary astrocytes (FIG. 6C). We confirmed long-term preservation of astrocytic identity by immunohistochemistry assessment of the expression of GFAP, S100B, VIM, ALDH1L1, and phalloidin at 2 months after induction (FIG. 6D). We did not detect any immunopositive cells in the control samples (data not shown, but see Supplementary FIG. 5 of Canals et al.). Immunopositive cells were rarely detected in controls infected only with rtTA, with the exception for VIM, which has been shown to be expressed in hES cells7 (data not shown, but see Supplementary FIG. 5 of Canals et al.). To confirm that our cultures consisted of highly enriched astrocytes and to exclude the presence of other cells, we analyzed markers of neural stem cells (Sox2 and Paired Box 6 (PAX6)), oligodendrocytes (myelin basic protein (MBP)), microglia (ionized calcium-binding adapter molecule 1 (Iba1)), neurons (Microtubule Associated Protein 2 (MAP2)), renal epithelia (atrial natriuretic peptide (ANP)) and skeletal muscle (myogenic differentiation 1 (MyoD)) at 14 and 21 days. We did not detect any immunopositive cells in any of the groups at these time-points (data not shown, but see Supplementary FIG. 5 of Canals et al.). Finally, we observed that iAs developed independent of doxycycline (Dox) treatment, and that regulation of endogenous Nfib and Sox9 was unaffected by transgenes after 7 days (FIG. 7A-C). The regulation of exogenous and endogenous expression of Nfib and Sox9 as well as dependence of doxycycline (Dox) for astrocytic phenotype of iAs was explored. As expected, in the presence of Dox, exogenous expression of Nfib and Sox9 was induced at 7-14 and 7 days, respectively. When Dox was removed at 7 or 14 days, exogenous Nfib and Sox9 diminished while Sox9 was downregulated even in the presence of Dox. On the other hand, endogenous expression of both Nfib and Sox9 increased at 7 days and remained similar at 14-21 days with or without Dox (FIG. 7A). Furthermore, removal of Dox at 7 or 14 days did not affect the percentage of cells expressing S100B or VIM at 21 days. Percentage of GFAP positive cells was reduced to around 20-30% when Dox was removed at 7 days but was unaffected when removed at 14 days (FIGS. 7B and 7C). This indicates that iAs becomes independent of Dox between 7 and 14 days, and that regulation of endogenous Nfib and Sox9 is not affected by the transgenes after 7 days. Our protocol yielded similar astrocytic morphology and immunophenotypes when applied to other pluripotent stem cell lines: H9 hESC and human induced pluripotent stem cells (hiPSC) (FIG. 8).

### Example 3

### Establishing Astrocytic Identity of the Induced Astrocytes

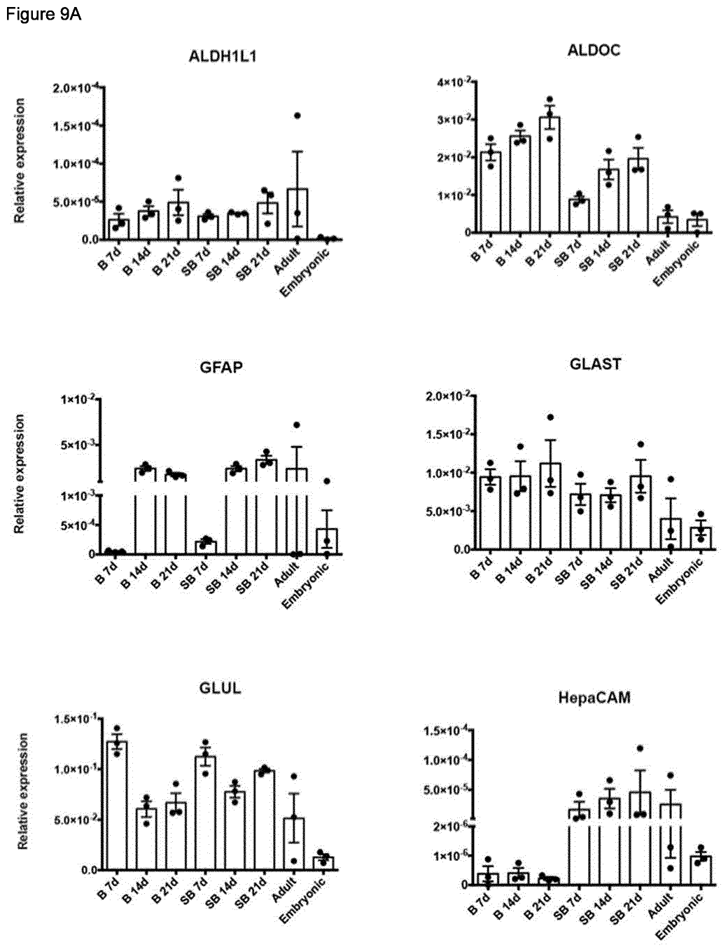

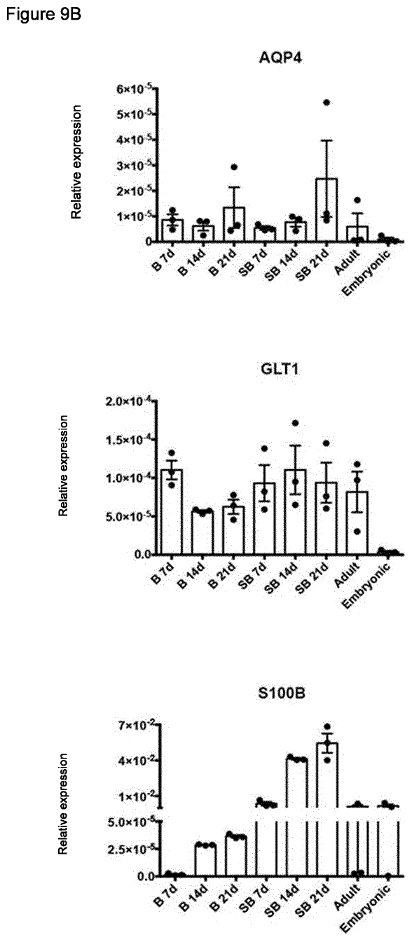

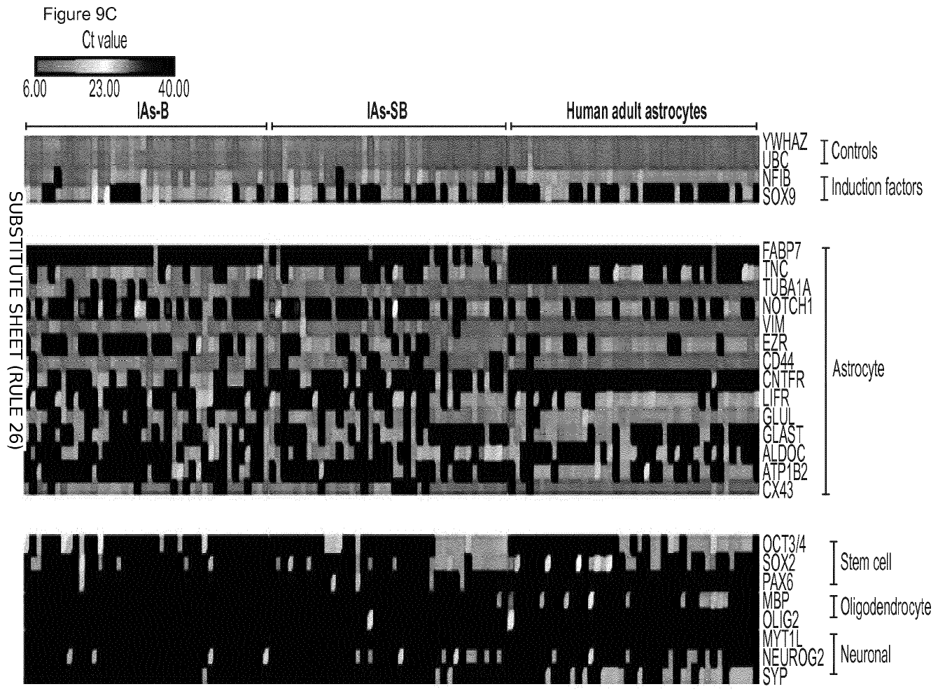

To provide further evidence for the astrocytic identity of iAs, we analyzed the expression of several astrocytic markers by RT—qPCR. We found that S100B and HEPACAM levels were higher in iAs-SB than in iAs-B and similar to those in adult human astrocytes, indicating that iAs-SB were more mature than iAs-B. Expression of other canonical astrocyte markers (ALDH1L1, ALDOC, AQP4, GFAP, GLAST, GLT1, GLUL HepaCAM, AND S100B) in iAs-B and iAs-SB closely resembled that in adult human astrocytes (FIGS. 9A and 9B). A multiplexed single-cell gene-expression analysis demonstrated that iAs-B and iAs-SB both had expression profiles similar to those of adult human astrocytes, with expression of several astrocytic genes such as GLUL, GLAST, TUBA1A, VIM, CD44, and GJA1 (also known as Cx43) (FIG. 9C). In contrast, pluripotency, neural stem cell, neuronal, and oligodendrocytic genes were not expressed or expressed at very low levels. This provides strong evidence that our iAs cultures were highly enriched in differentiated astrocytes. Taken together, these findings demonstrate that iAs-B and iAs-SB display cardinal astrocyte markers and morphology and can be generated rapidly and efficiently from different pluripotent stem cell lines.

### Example 4

Functional characterization of induced astrocytes

To address whether the iAs could perform astrocytic functions their ability to clear neurotransmitters was evaluated. Glutamate uptake assays 14 days after induction showed that iAs took up glutamate to an extent similar to that of human astrocytes (FIG. 1G).

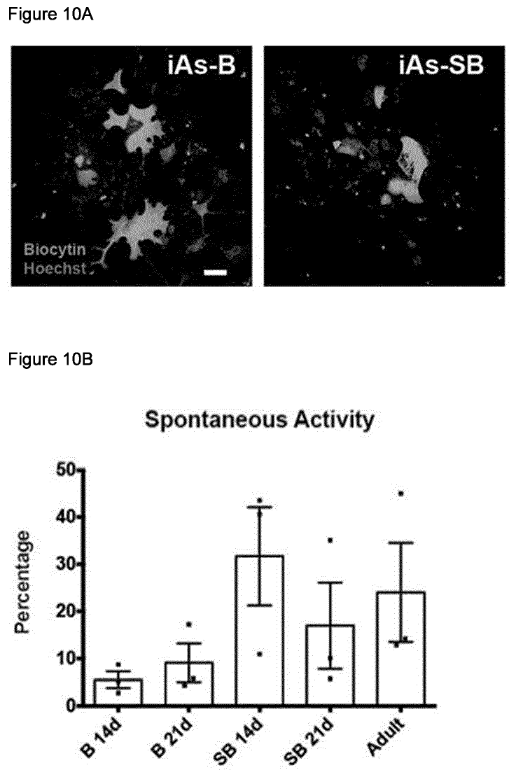

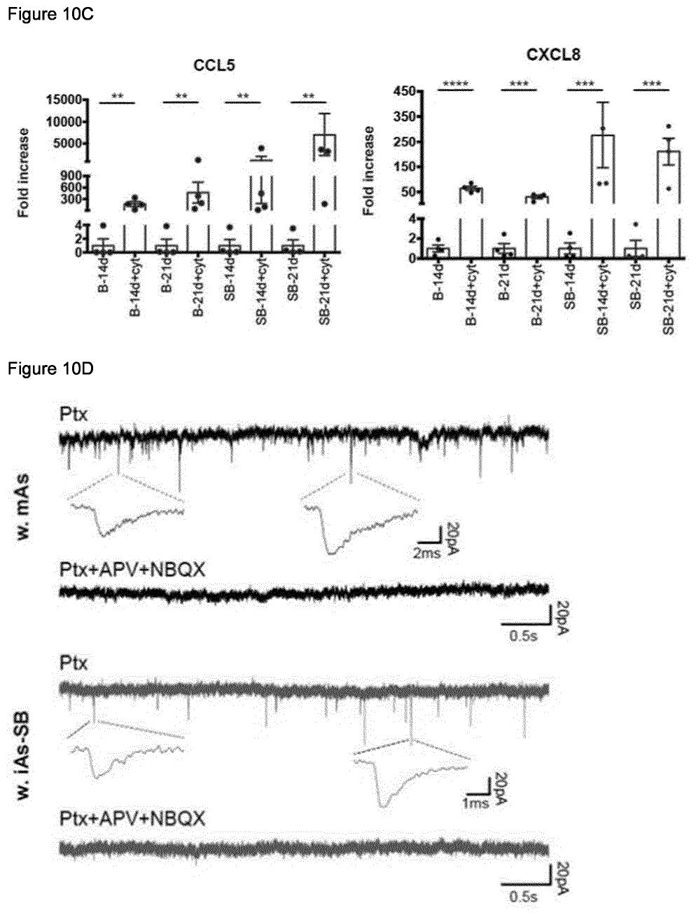

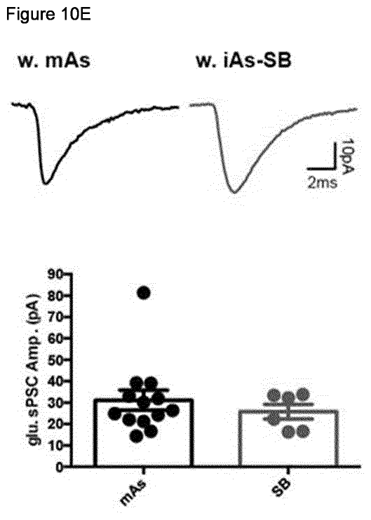

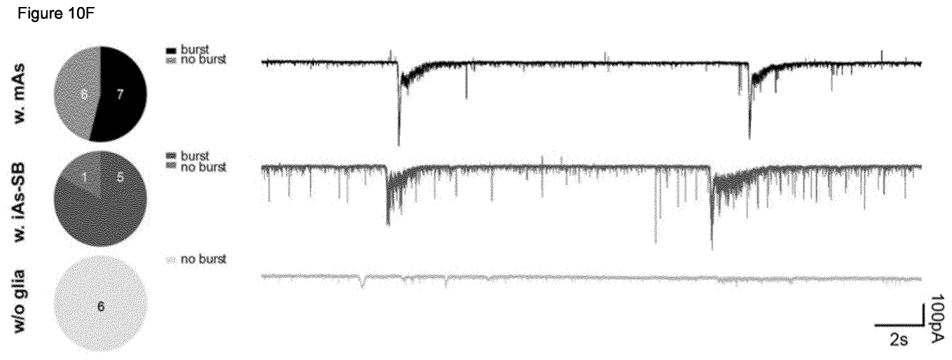

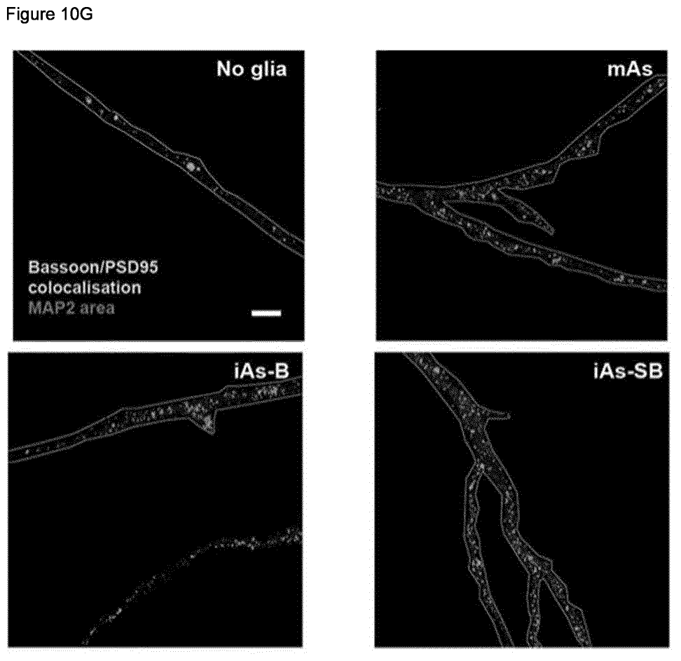

A structural property of astrocytes is the formation of gap junctions that can be detected by the propagation of biocytin. We filled single cells with biocytin and observed diffusion into neighboring astrocytes, which confirmed the presence of functional gap junctions (FIG. 1H and FIG. 10A).

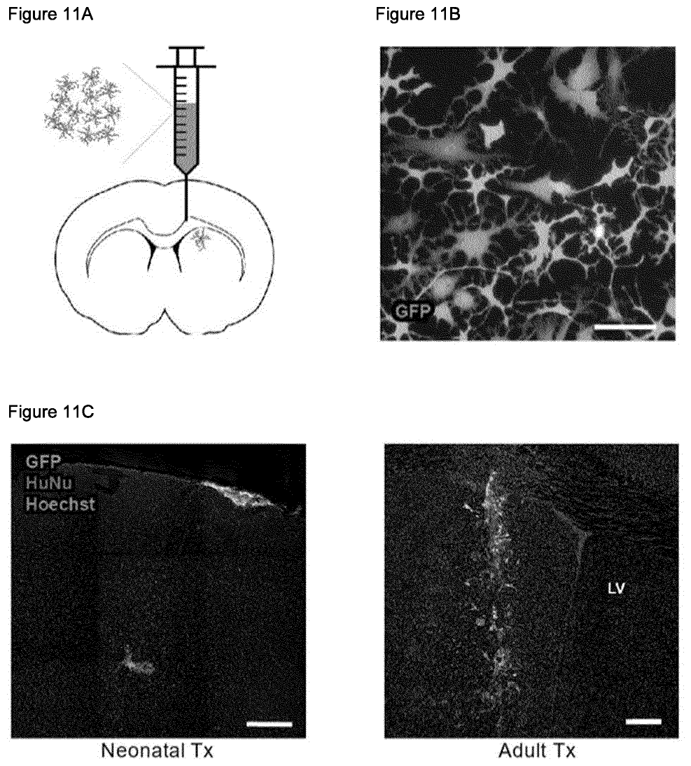

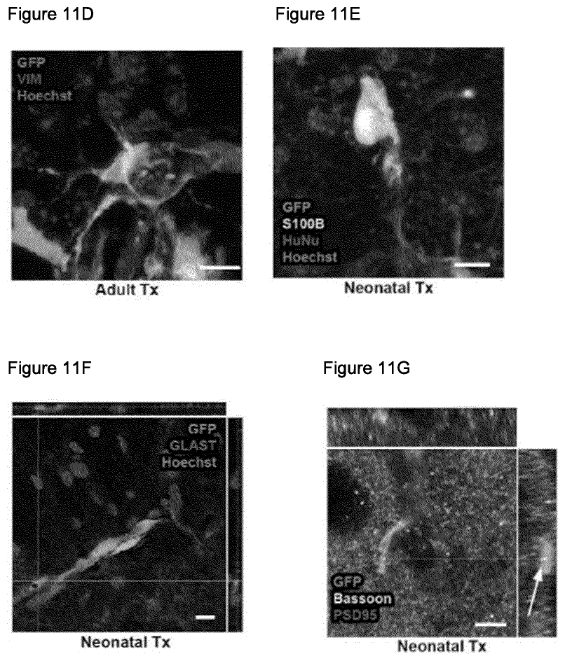

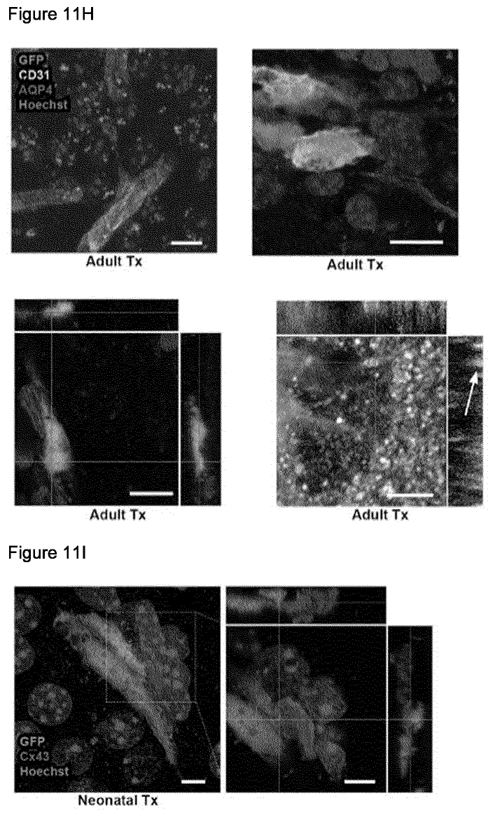

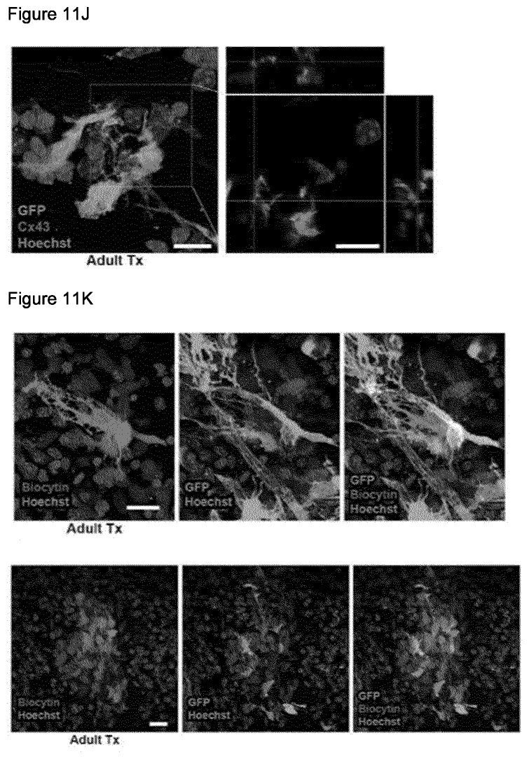

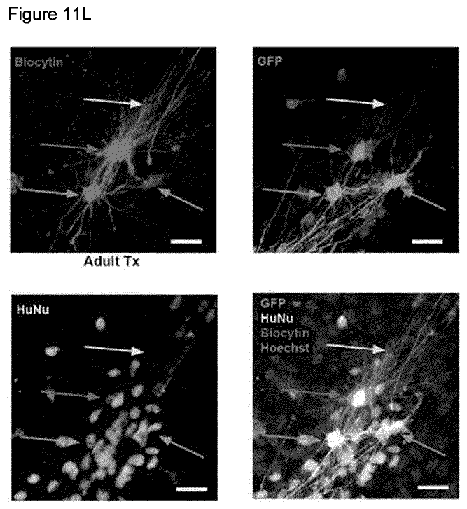

Another well-described property of astrocytes is a spontaneous or stimulated increase in the amount of intracellular calcium. Using calcium imaging, we found a population that displayed spontaneous calcium waves at 14 and 21 days after induction (iAs-B, 5-9%; iAs-SB, 17-31%), at levels similar to that of adult human astrocytes (24%) (FIG. 10B). Moreover, most iAs responded to ATP at 14 and 21 days after induction iAs-B, 80-85%; iAs-SB, 75-94%), to a similar degree as adult human astrocytes (84%) (FIG. 11).

Astrocytes are important in neuroinflammation. Therefore, we exposed iAs to 1L-1β and assessed the expression of IL-6, CXCL8, CXCL10, and CCL5, all of which have been reported to be induced in human primary astrocytes. Notably, iAs showed increased expression of these cytokines (FIG. 1J and FIG. 10C), and iAs-SB displayed a stronger response, supporting their higher degree of maturation.

### Example 5

### Induced Astrocytes Promote Synapse Formation

The ability of human-derived iAs to support synapse formation in human induced neurons (iNs) similarly to primary mouse astrocytes (mAs) was assessed. We co-cultured iNs on iAs and analyzed the occurrence of spontaneous postsynaptic currents (sPSCs). On iAs-SB, 75% of iNs expressed sPSCs with frequencies similar to those on mAs (FIG. 1K). In contrast, only 55% of iNs expressed sPSCs when cultured without glia, and the frequency of the sPSCs was lower than those in iNs co-cultured with iAs-SB (FIG. 1L, FIGS. 10D and 10E). To characterize the sPSCs in more detail, we isolated the glutamatergic sPSCs by addition of picrotoxin (Ptx), showing that the amplitude of glutamatergic sPSCs was similar for iNs co-cultured with mAs and iAs-SB, and that the glutamatergic sPSCs were blocked in the presence of the NMDA and AMPA receptor antagonists, D-APV and NBQX (FIGS. 10D and 10E). These findings clearly demonstrated the occurrence of functional glutamatergic synapses. We also observed burst-like activity in iNs co-cultured with either mAs (54%) or iAs-SB (83%), which was not detected in iNs without glia (FIG. 10F). Moreover, co-staining for the pre- and postsynaptic proteins Bassoon and PSD95, respectively, showed that iNs without co-culture exhibited low amounts of Bassoon and PSD95 co-staining in their neurites. In contrast, in iNs that were co-cultured with iAs-B or iAs-SB, 16% and 14% of the MAP2+ neurite area was covered by Bassoon and PSD95 co-staining, respectively (FIG. 1M and FIG. 10G), similar to what was observed after co-culture with mAs (15%). Thus, co-culture with iAs increased synapse formation compared with that in iNs alone, confirming the ability of iAs to support synapse formation.

Taking Examples 4 and 5 together, our findings in five different assays provide strong evidence that iAs exhibit all major functional properties of human astrocytes.

### Example 6

### Induced Astrocytes are Implantable

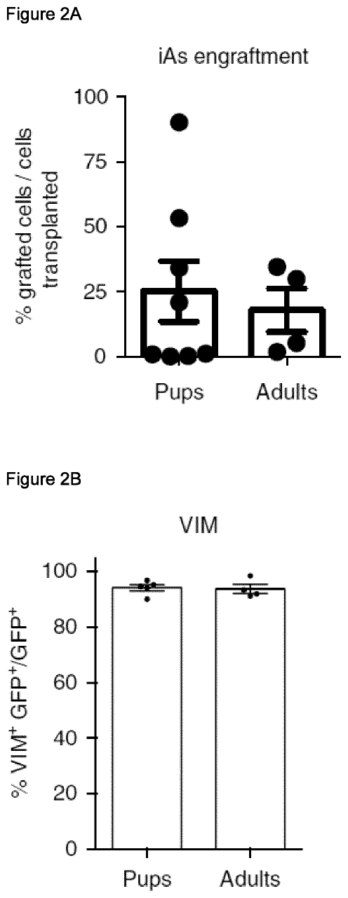

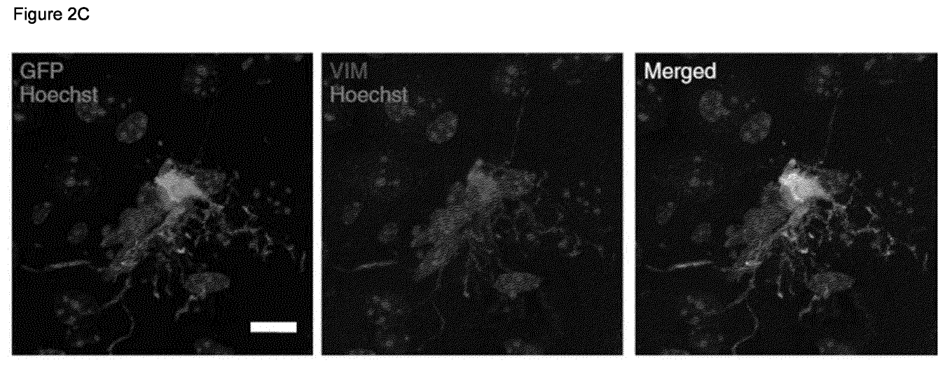

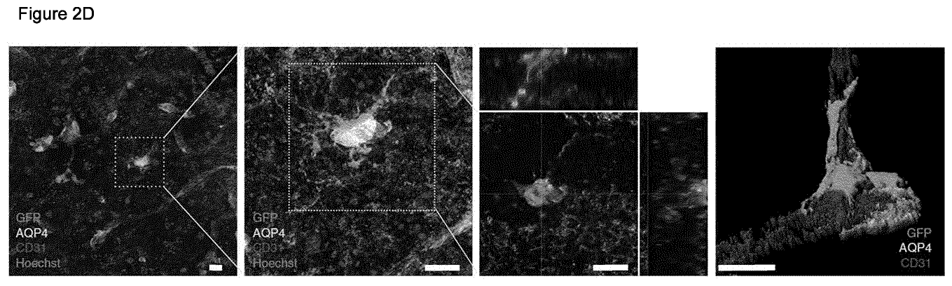

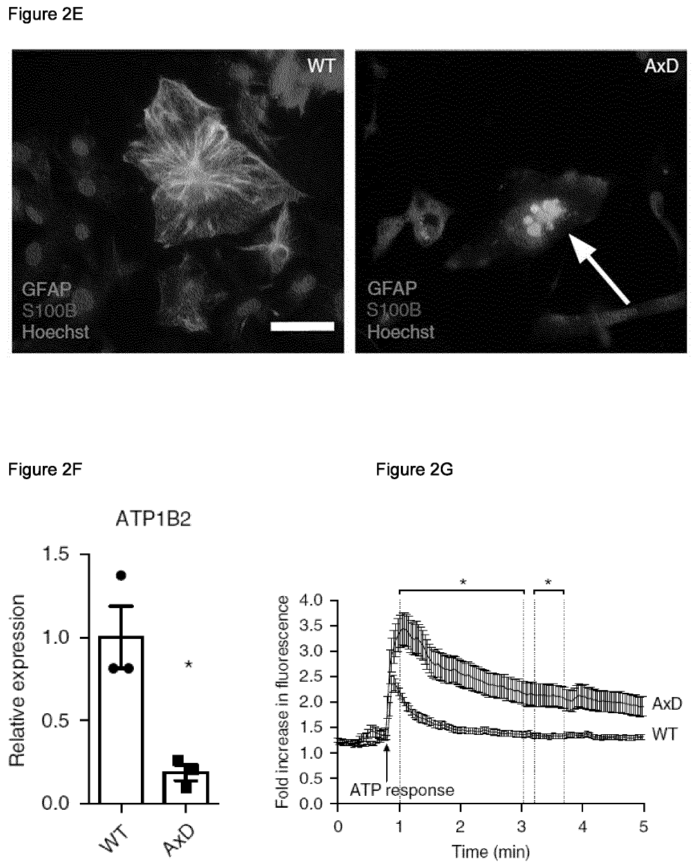

Whether iAs survived intracerebral implantation was assessed. It was found that grafted iAs-SB localized in cores or as isolated cells (FIG. 11A-C). About 25% and 18% of implanted cells survived in neonatal and adult mice, respectively (FIG. 2A), and >90% retained VIM expression (FIGS. 2C, 2B and FIG. 11D). The grafted cells stained positive for S100B and GLAST, and GFP+astrocytic processes co-localized with Bassoon⁺ and PSD95⁺ synaptic structures on host neurons (Supplementary FIG. 11E-G). We found iAs in close proximity to vessels with AQP4⁺ processes encasing CD31+ blood vessels (FIG. 2D and FIG. 11H) and Cx43⁺ connections with neighboring cells (FIGS. 11I and 11J). Furthermore, iAs propagated biocytin to neighboring grafted and host cells in acute slices (FIGS. 11K and 11L). These results strongly indicate that implanted iAs retain an astrocytic phenotype and are functionally integrated in the mouse brain.

### Example 7

### Induced Astrocytes are Useful in Neurological Disease Modeling

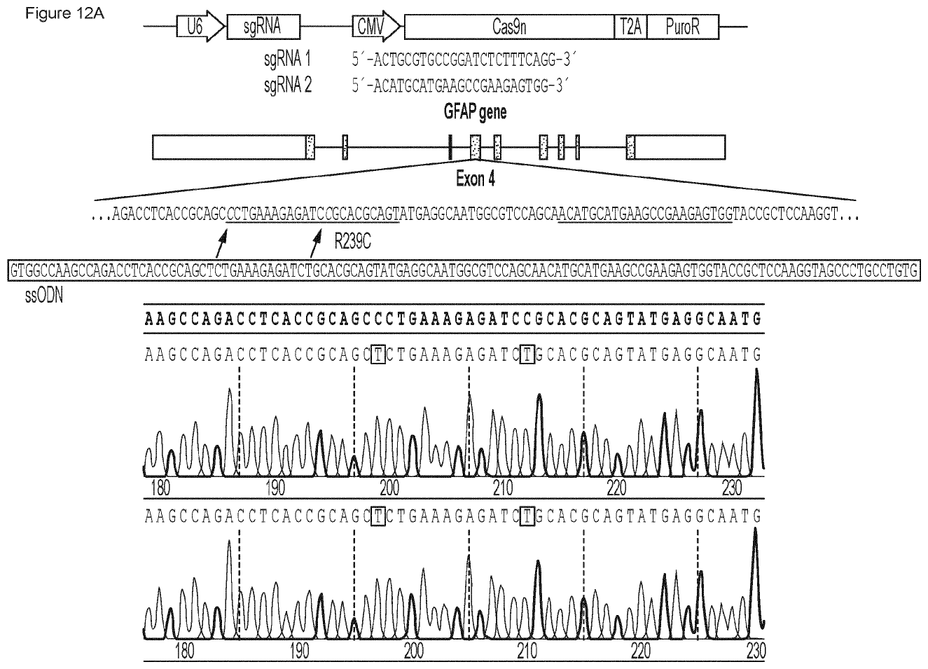

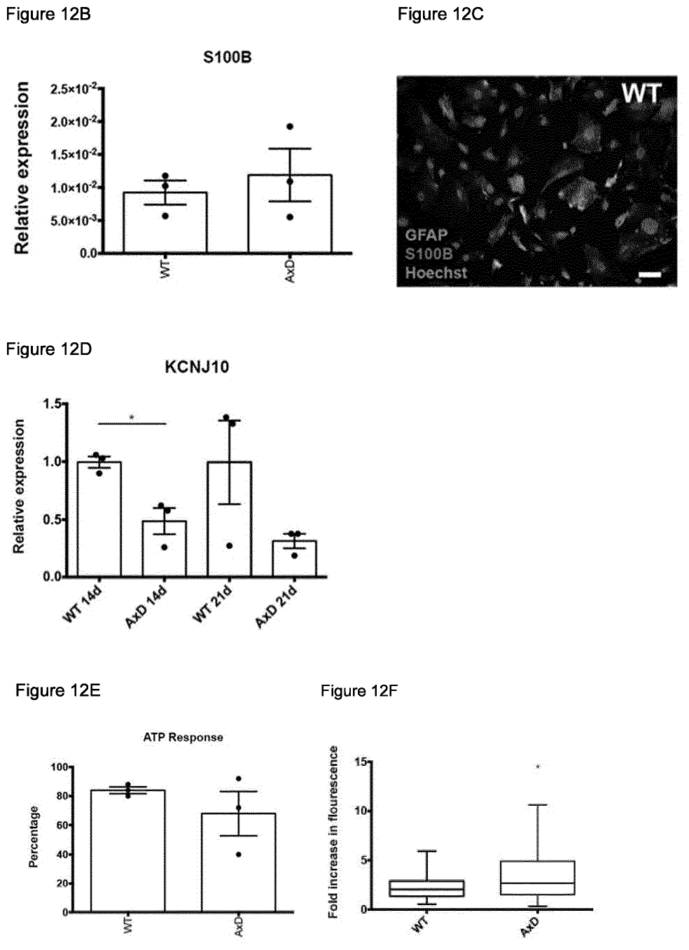

Alexander disease (AxD) is a leukodystrophy caused by mutations in GFAP and is characterized by the presence of aggregated GFAP. To model AxD, we introduced an R239C mutation into H1 hESCs by CRISPR—Cas9 gene editing followed by iAs-SB induction (FIGS. 12A). Plasmids containing two different sgRNAs targeting regions near the mutation site, the nickase version of Cas9 (Cas9n) and a DNA repair template (ssDNA) containing the AxD mutation as well as a silent mutation in the PAM (protospacer adjacent motif) sequence were delivered to H1 cells by transient transfection (FIG. 12A). Following selection, individual clones were picked and expanded. The presence of the AxD mutation (FIG. 12A) and absence of unwanted editing (data not shown) in isolated clones were confirmed by PCR amplification and sequencing of the region of interest as well as of top predicted off-target sites in chromosome 11, 16, and 18 for sgRNA A and in chromosome 4, 10, and 20 for sgRNA B. Sequencing chromatograms included 40 nucleotides up-stream and down-stream of the target sequence. We did not detect any difference in astrocyte induction efficiency between wild-type (WT) and AxD iAs on the basis of S100B expression (FIG. 12B). Notably, we found GFAP inclusions in AxD iAs similar to those described previously, which were not detected in the controls (FIG. 2E and FIG. 12D). Furthermore, AxD iAs exhibited reduced expression of the Na+/K+ ATPase ATP1B2 and the potassium channel KCNJ10 (FIG. 2F and FIG. 12D), which have previously been described in AxD19. Abnormalities in calcium signaling were recently reported in a mouse model of AxD20. Using calcium imaging, we detected no differences between WT and AxD iAs in terms of the percentage of cells that responded to ATP (FIG. 10E). However, we found a greater increase in the amount of intracellular calcium, together with a slower recovery to basal levels, in AxD iAs compared with that in WT cells (FIG. 2G and FIG. 12F). The results show that iAs can recapitulate the structural and functional disease phenotypes in AxD.

### Example 8

### Experimental Methods

### Cell Culture.

H1 (WA01) cells, H9 (WA09) hESCs from WiCell Research Institute (Wicell, Wiss.), and iPSCs (clone RB9-CB1 (Rönn, R. E. et al. Stem Cell Rep. 4:269-281 (2015)); kindly provided by Niels-Bjarne Woods (Lund University)) were cultured in feeder-free conditions using mTeSR1 medium (StemCell Technologies) on MATRIGEL-coated six-well plates (Corning), with medium changed daily. Cells were dissociated with Accutase (Thermo Fisher Scientific) when the culture reached ˜80% confluency and replated in mTeSR1 supplemented with 500 nM thiazovivin (Sigma-Aldrich) during the first 24 h.

For human adult astrocytes, fresh cortical tissue was obtained during resection surgery from patients who had pharmacologically intractable epilepsy. The use of human brain tissue was approved by the local ethical committee in Lund (212/2007) and was carried out in accordance with the Declaration of Helsinki. Prior to each surgery, written informed consent was obtained from all of the subjects. The samples were submerged in Hibernation medium (Thermo Fisher Scientific) immediately after surgery. Meninges were removed under a dissection stereomicroscope (Leica), and the tissue was chopped into small chunks with a sterile surgical blade and processed with the Adult Brain Dissociation Kit (Miltenyi Biotec) according to the manufacturer's instructions. A single-cell suspension was obtained and plated onto poly-d-lysine (PDL) and human-fibronectin-coated culture vessels (Thermo Fisher Scientific). Cells were cultured in Neurobasal medium supplemented with B27, Glutamax (Thermo Fisher Scientific), brain derived neurotrophic factor (hBDNF; Peprotech), glial-cell-derived neurotrophic factor (GDNF; Peprotech), and ciliary neurotrophic factor (CNTF; Peprotech). After 3-4 weeks of cultivation, the medium was switched to DMEM:F12 1:1 supplemented with the N2 and G5 supplements (Thermo Fisher Scientific).

For human-fetus-derived astrocytes, cortical tissue from dead, aborted human fetuses, aged 7-9 weeks after conception, was obtained from Lund and Malmo University Hospitals according to guidelines approved by the Lund/Malmo Ethical Committee (2017/1). Prior to abortion, written informed consent was obtained from all of the subjects. Fetuses were kept in Hibernation medium, and dissection was conducted under a stereomicroscope (Leica). The central nervous system was isolated and cleaned from the surrounding tissue. The cortex was cut open along and close to the dorsal midline and dissected out. To generate primary astrocytic monolayer cultures, we incubated the cortex in Neurobasal supplemented with B27 and Glutamax, and mechanically dissociated the tissue until a single-cell suspension was obtained. Cells were plated onto PDL- and laminin-coated dishes in DMEM:F12 1:1 supplemented with N2 and 10% fetal bovine serum (all from Thermo Fisher Scientific).

### Lentiviral Constructs and Virus Production.

Full-length cDNAs of mouse Nfia, Nfib, and Sox9 genes was amplified from plasmids available at Addgene (#64901, #64900, and #41080, respectively), and specific restriction sites were added to allow cloning in tetO-FUW (tetracycline operator FUW) lentiviral vectors carrying genes for resistance to blasticidin, hygromycin (Addgene #97330), and puromycin (Addgene #97329) with EcoRI/BamHI (Nfia and Nfib) or EcoR1/Xbal (Sox9) restriction enzyme combinations. The rtTA (reverse tetracycline-controlled transactivator) and tetO-FUW-GFP lentiviral vectors were obtained from Addgene (#20342 and #30130, respectively), and the GFAP::GFP vector was a kind gift from Chun-Li Zhang (University of Texas Southwestern Medical Center). The primers used in the cloning process are shown in Table 2. For the lentiviral production protocol, see Example 9.

TABLE 2
Primers used for cloning the factors
into the lentiviral vectors with selection
genes.

| Primer name | Primer Sequence |
|---|---|
| Sox9 Forward | 5′-GAATTCATGAATCTCCTGGACCCCTTC-3′ |
| Sox9 Reverse | 5′-TCTAGAGGGTCTGGTGAGCTGTGTG-3′ |
| Nfia Forward | 5′-GAATTCCATGTATTCTCCGCTCTGTCTCAC-3′ |
| Nfia Reverse | 5′-GGATCCTTATCCCAGGTACCAGGACTG-3′ |
| Nfib Forward | 5′-GAATTCATGATGTATTCTCCCATCTGTCTCAC-3′ |
| Nfib Reverse | 5′-GGATCCCTAGCCCAGGTACCAGGAC-3′ |

Generation of iAs From hESCs and iPSCs.

On day −2, H1, H9 hESCs, and human iPSCs at ˜80% confluency were dissociated with Accutase, and 5×105 cells were replated in MATRIGEL-coated six-well plates using mTeSR1 (StemCell technologies) medium with 10 μM ROCK (p160-Rho-associated coiled kinase) inhibitor (Y-27632; StemCell Technologies). One day later (day −1), medium was replaced by fresh mTeSR1 medium with 8 μg/ml polybrene (Sigma-Aldrich), and 1 μl of each virus was added per well. One day after infection (day 0), medium was replaced with fresh mTeSR1 medium containing 2.5 μg/ml doxycycline, which was kept in the medium throughout the experiments. On days 1 and 2, cells were cultured in Expansion medium (DMEM/F-12, 10% FBS, 1% N2 supplement, and 1% Glutamax, from Thermo Fisher Scientific). From day 3 to day 5, Expansion medium was gradually switched to FGF medium (Neurobasal, 2% B27 supplement, 1% NEAA, 1% Glutamax, and 1% FBS, from Thermo Fisher Scientific; 8 ng/ml FGF, 5 ng/ml CNTF, and 10 ng/ml BMP4, from Peprotech), with the exception of the case of AxD modeling, for which serum was kept in the medium to enhance GFAP expression. On day 6, the mixed medium was replaced by FGF medium. Selection was carried out on days 1-5 for vectors that rendered cells resistant to blasticidin (1.25 μg/ml) or hygromycin (200 μg/ml) and on days 1-2 for vectors that made cells resistant to puromycin (1.25 μg/ml). On day 7, cells were dissociated with Accutase and replated in MATRIGEL-coated wells or coverslips. The day after, FGF medium was replaced, and afterward 50% of the medium was replaced by Maturation medium (1:1 DMEM/F-12 and Neurobasal, 1% N2, 1% sodium pyruvate, and 1% Glutamax, from Thermo Fisher Scientific; 5 mg/ml N-acetyl-cysteine, 500 mg/ml dbcAMP, from Sigma-Aldrich; 5 ng/ml heparin-binding EGF-like growth factor, 10 ng/ml CNTF, 10 ng/ml BMP4, from Peprotech) every 2-3 d, and cells were kept for 14, 21, or 28 d. The detailed composition of each medium can be found in Example 9.

Coculture of iAs with Induced Neurons.

hESC iNs were produced via a previously described protocoll4. On day 7 of both protocols, iNs and iAs were dissociated with Accutase and replated together on poly-d-lysine-(Sigma) and laminin-coated (Sigma) coverslips (50,000 iAs with 100,000 iNs). Cells were kept for 32-35 additional days in culture medium consisting of 1:1 Maturation medium and Neuronal medium consisting of BrainPhys (StemCell Technologies), 1% N2 supplement, 2% B27 supplement, 10 ng/ml BDNF (Peprotech), and 10 ng/ ml NT-3 (Peprotech).

For quantification of Bassoon—PSD95 co-staining, images of neurons were acquired with a Zeiss LSM 780 confocal microscope with a 63× objective lens and analyzed with Zen software. Laser power and photomultiplier gain were set at the same levels for all of the samples to allow for quantitative comparisons. Five neurons per coverslip were analyzed from a total of seven coverslips from two independent experiments for each sample. Co-localizing puncta inside the MAP2 region were detected with the co-localization threshold plug-in of Fiji. We quantified the number of puncta inside the MAP2 region by using the particle analysis plug-in of ImageJ and counting puncta >0.1 μm in size to discard background.

### Immunofluorescence.

All primary and secondary antibodies used can be found in Tables 3 and 4. For immunocytochemical analysis, cells were plated on MATRIGEL-coated 13-mm-diameter glass coverslips or in MATRIGEL-coated 24-well plates. At the indicated times, cells were washed one to three times with potassium-phosphate-buffered saline (KPBS) and fixed for 20 min at room temperature (RT) in 4% paraformaldehyde (PFA), washed three times with KPBS and blocked for 60 min in KPBS containing 0.025% Triton X-100 (TKPBS) and either 5% normal donkey serum (NDS; Millipore) or 2.5% NDS and 2.5% normal goat serum (NGS; Millipore). Primary antibody incubation was performed overnight at 4° C. in blocking solution. Cells were then washed twice for 5 min with 0.025% TKPBS and once for 5 min with blocking solution.

Incubation with secondary antibodies or phalloidin-TRITC was performed in blocking solution for 2 hr at RT. Cells were then washed once with 0.025% TKPBS for 5 min and twice for 5 min with KPBS, and coverslips were mounted using PVA:DABCO, polyvinyl alcohol (Sigma-Aldrich)-based mounting media containing DABCO (Sigma-Aldrich) anti-fading reagent.

TABLE 3
Primary antibodies

| | | | Dilu- | Dilu- |
|---|---|---|---|---|
| | | | tion | tion |
| Antibody | Vendor | Catalog # | ICC | IHC |
| gp α-GFAP | Synaptic Systems | 173004 | 1:500 | 1:500 |
| rbb α-5100β | Dako | Z0311 | 1:400 | 1:400 |
| ms α-Vimentin | Dako | M0725 | 1:250 | 1:250 |
| rbb α-ALDH1L1 | Novus Biologicals | NBP2-25143SS | 1:1000 | |
| ms α-Ki67 | Abcam | Ab16667 | 1:100 | |
| ms α-HuNu | Millipore | MAB1281 | | 1:100 |
| rbb α-Sox2 | Millipore | AB5603 | 1:200 | |
| ms α-MBP | Covance | SMI-99P | 1:1000 | |
| g α-Sox10 | SantaCruz | sc-17343 | 1:100 | |
| ch α-MAP2 | Abcam | ab5302 | 1:10000 | |
| rbb α-GLAST | Abcam | ab416 | 1:200 | 1:200 |
| ms α-PSD95 | Abcam | ab2723 | 1:200 | 1:100 |
| rbb α-Bassoon | Synaptic Systems | 141 013 | 1:1000 | 1:1000 |
| g α-GFP | Abcam | ab5450 | 1:2000 | |
| rbb α-Cx43 | SantaCruz | sc-9059 | 1:100 | 1:100 |
| rbb α-AQP4 | Sigma-Aldrich | A5971 | 1:100 | 1:100 |
| rat α-CD31 | BD Biosciences | 550274 | 1:400 | |
| ms α-ANP | R&D Systems | MAB3815 | 1:100 | |
| ms α-MyoD | BD Biosciences | BD554130 | 1:250 | |
| rbb α-Iba1 | Wako | 019-19741 | 1:1000 | |

| | | | |
|---|---|---|---|
| ms α-VIM-PE | BD Biosciences | 562337 | FACS 1:100 |
| ms α-CD44-BV510 | BD Biosciences | 563029 | FACS 1:100 |

ICC = immunocytochemistry.
IHC = immunohistochemistry.

TABLE 4
Secondary antibodies

| | | | Dilu- | Dilu- |
|---|---|---|---|---|
| | | | tion | tion |
| Antibody | Vendor | Catalog # | ICC | IHC |
| Goat α-guinea pig AF488 | Life | A11073 | 1:500 | |
| | Technologies | | | |
| Donkey α-mouse AF488 | Life | A21202 | 1:500 | |
| | Technologies | | | |
| Donkey α-mouse AF568 | Life | A10037 | 1:500 | 1:250 |
| | Technologies | | | |
| Donkey α-rabbit AF488 | Life | A21206 | 1:500 | |
| | Technologies | | | |
| Donkey α-rabbit AF568 | Life | A10042 | 1:500 | 1:250 |
| | Technologies | | | |
| Donkey α-rabbit AF647 | Life | A31573 | | 1:250 |
| | Technologies | | | |
| Donkey α-goat AF488 | Life | A11055 | 1:500 | 1:250 |
| | Technologies | | | |
| Donkey α-chicken Cy3 | Jackson | 703-165-155 | 1:500 | |
| | ImmunoRes | | | |
| Donkey α-chicken AF647 | Jackson | 703-605-155 | 1:500 | |
| | ImmunoRes | | | |
| Donkey α-rat AF647 | Jackson | 712-605-153 | | 1:250 |
| | ImmunoRes | | | |
| Phalloidin-TRITC | Sigma- | P1951 | 2.5 μg/ml | |
| | Aldrich | | | |
| Streptavidin AF568 | Life | S11226 | 1:500 | 1:200 |
| | Technologies | | | |
| Streptavidin AF488 | Life | S11223 | 1:500 | |
| | Technologies | | | |

ICC = immunocytochemistry.
IHC = immunohistochemistry.

For immunohistochemical analysis, sections were washed three times with KPBS and blocked for 60 min in 0.25% TKPBS containing 5% NDS, or 2.5% NDS and 2.5% NGS. Primary antibody incubation was performed overnight at 4° C. in blocking solution. Sections were then washed twice with 0.25% TKPBS and once with blocking solution. Incubation with secondary antibodies was performed in blocking solution for 2 hr. at RT. Sections were then washed once with 0.25% TKPBS and twice with KPBS before being mounted on slides and covered with a coverslip using PVA:DABCO. For GLAST and Cx43 staining, all washes and incubations with antibodies were done in KPBS, and the blocking step was extended to 2 h. All washes were done for 10 min, and all of the steps were performed on an orbital shaker.

For nuclear staining, 1 μg/ml Hoechst (Thermo Fisher Scientific) was used during the incubation with the secondary antibody.

Images were obtained with a Zeiss LSM 780 confocal microscope, orthogonal reconstruction was done with Zen software, and 3D rendering was done with Imaris (Bitplane).

### FACS.

Cells were dissociated with Accutase, pelleted for 5 min at 300 g, washed in PBS, and pelleted again for 5 min at 300 g. For CD44, cells were resuspended in 100 μl of FACS buffer containing PBS +1% FBS +0.09% sodium azide. Cells were then incubated for 30 min at RT in the dark with antibody and 7AAD for viability and then washed twice with FACS buffer and pelleted for 5 min at 300 g. Before analysis, cells were resuspended in 250 μl of FACS buffer. For VIM, after dissociation, cells were treated with a Fixation/Permeabilization Solution Kit (BD Biosciences) according to the manufacturer's instructions. For analysis, samples were run in a BD FACS LSRFortessa analyzer, and the data were analyzed with FlowJo software.

### Glycogen Staining.

Cells on coverslips were washed once with cold KPBS and fixed with cold methanol for 5 min. After fixing, cells were washed three times with 70% ethanol and then incubated for 30 min in 1% periodic acid (Sigma-Aldrich) diluted in 70% ethanol. After three washes with 70% ethanol, cells were stained with 0.5% Basic Fuchsin (Sigma-Aldrich) diluted in acid ethanol (8.0 parts absolute ethanol, 1.9 parts water, and 0.1 parts HCl 37%). After staining, cells were washed three times with 70% ethanol and once with KPBS, and mounted with PVA:DABCO.

### Cell Quantification.

In vitro cell counting was performed under a BX61 epifluorescence microscope (Olympus) in 20 random fields of view from three replicates for each condition and time point, and the counts were normalized to the number of Hoechst+ cells. In vivo quantification was performed in all sections containing iAs. The total number of GFP+ iAs and co-localization with VIM were assessed.

### Gene Expression.

RNA isolation was performed with the RNeasy Mini Kit (Qiagen) according to the manufacturer's instructions. To prevent DNA contamination, RNA was treated with DNase I (Qiagen). The yield of RNA was determined with a Nanodrop ND-1000 spectrophotometer (Saveen & Werner). One microgram of RNA was reverse-transcribed with the qScript cDNA Synthesis Kit (Quantabio). For mouse and human NFIB and SOX9 analysis, RT-qPCR was carried out with Fast SYBR Green Master Mix (Thermo Fisher Scientific) and specific primers for each gene (Table 5) with a StepOnePlus Real-Time PCR System (Thermo Fisher Scientific). Other RT-qPCRs were carried out with TaqMan Universal PCR Master Mix and TaqMan assays (Table 6) with an iQ5 Real-Time PCR detection system (Bio-Rad). For single-cell qR-PCR analyses, viable (as determined by DRAQ7 staining) iAs or primary astrocytes were sorted through a FACSAria II (BD Biosciences) to individual wells of 96-well plates, and mRNA levels were quantified with the Fluidigm Biomark dynamic array system as previously described (Yang, N. et al. Nat. Methods 14:621-628 (2017)). Data can be presented as heat maps for example as generated by the Morpheus online tool (clueDOTio/Morpheus/).

TABLE 5
Primers for NFIB and SOX9 RT-qPCR

| Primer name | Primer sequence |
|---|---|
| mNfib Forward | 5′-GACATGAACTCTGGTGTGAACCTG-3′ |
| mNfib Reverse | 5′-GTAGTTGGAGAAGACATATCTTGATCTC-3′ |
| hNFIB Forward | 5′-GACATGAACTCGGGGGTCAATCTT-3′ |
| hNFIB Reverse | 5′-GTAGTCGGAGAAGACATATCTTGATC-3′ |
| mSox9 Forward | 5′-GCGAGCACTCTGGGCAATCTC-3′ |
| mSox9 Reverse | 5′-CTGCCCCCCTCTGCCAGA-3′ |
| hSOX9 Forward | 5′-CGAGCACTCGGGGCAATCC-3′ |
| hSOX9 Reverse | 5′-CTGCCCCCCTCTGGCAAG-3′ |
| GAPDH Forward | 5′-GCACCGTCAAGGCTGAGAAC-3′ |
| GAPDH Reverse | 5′-AGGGATCTCGCTCCTGGAA-3′ |

TABLE 6
TaqMan assays

| Gene | Assay ID |
|---|---|
| ALDH1L1 | Hs00201836_m1 |
| AQP4 | Hs00242342_m1 |
| ALDOC | Hs00902799_g1 |
| ATP1B2 | Hs00155922_m1 |
| CCL5 | Hs00982282_m1 |
| CD44 | Hs01075862_m1 |
| CNTFR | Hs00181798_m1 |
| CXCL8 | Hs00174103_m1 |
| CXCL10 | Hs00171042_m1 |
| EZR | Hs00931646_m1 |
| FABP7 | Hs00361426_m1 |
| GAPDH | Hs02758991_g1 |
| GFAP | Hs00909233_m1 |
| CX43 | Hs00748445_s1 |
| GLUL | Hs00365928_g1 |
| HEPACAM | Hs00404147_m1 |
| IL-6 | Hs00174131_m1 |
| LIFR | Hs01123581_m1 |
| MBP | Hs00921945_m1 |
| MYT1L | Hs00903951_m1 |
| NEUROG2 | Hs00702774_s1 |
| NFIB | Hs01029174_m1 |
| NOTCH1 | Hs01062014_m1 |
| OCT4 | Hs01654807_s1 |
| OLIG2 | Hs00377820_m1 |
| PAX6 | Hs00240871_m1 |
| GLT1 | Hs01102423_m1 |
| GLAST | Hs00188193_m1 |
| SOX2 | Hs01053049_s1 |
| SOX9 | Hs00165814_m1 |
| SYP | Hs00300531_m1 |
| S100B | Hs00902901_m1 |
| TNC | HS01115665_m1 |
| TUBA1A | Hs00362387_m1 |
| UBC | Hs00824723_m1 |
| VIM | Hs00958111_m1 |
| YWHAZ | Hs03044281_g1 |

### Calcium Imaging.

Cells cultured in MATRIGEL-coated 35-mm glass-bottom Petri dishes (Ibidi) were loaded for 30 min with 2 μM Fluo-4 (calcium indicator; Life Technologies) prepared according to the manufacturer's instructions. Cells were washed with medium once and imaged immediately. Live fluorescence imaging was done with a Zeiss LSM 780 confocal microscope, and images were taken every 1.94 s with a 10× objective for 5 min before and 15 min after the addition of ATP (100 μM or 30 μM in AxD calcium-imaging assays). For quantification of the change in intensity over time, astrocytes were outlined as regions of interest (ROIs) and analyzed with Zen software (Zeiss). For each ROI, the change in fluorescence intensity over time was plotted. The percentage of activated cells was counted manually, with ROIs displaying transients (increasing fluorescence values at least 50% of the basal value) in each video compared with the total number of ROIs.

### Glutamate Uptake.

Cells were plated on days 6-7 in MATRIGEL-coated six-well plates and cultured until day 14 or 21. After 30 min of incubation in Hank's balanced salt solution (HBSS) buffer without calcium and magnesium (Gibco), cells were incubated for 3 h in HBSS with calcium and magnesium (Gibco) containing 100 μM glutamate. Samples of medium were collected after 3 h and analyzed with a colorimetric glutamate assay kit (Sigma-Aldrich) used according to the manufacturer's instructions.

Cytokine Stimulation.

Induced astrocytes differentiated for 14 or 21 days were incubated for 8 h with or without 10 ng/ml IL-1β (Peprotech) in fresh Maturation medium. After stimulation, RNA isolation and real-time qPCR were performed as described above.

### Electrophysiology.

Co-cultures of iNs with mAs or iAs-SB, or without glia, were grown on coverslips (see above) and transferred to the recording chamber for in vitro recordings. During electrophysiological recordings, coverslips were constantly perfused with carbogenated artificial cerebral spinal fluid (ACSF; 119.0 mM NaCl, 2.5 mM KCl, 1.3 mM MgSO4, 2.5 mM CaCl2, 26.0 mM NaHCO3, 1.25 mM NaH2PO4, and 11.0 mM glucose, pH ˜7.4) at 34° C. Whole-cell patch-clamp recordings from iNs were performed using a cesium-based intracellular solution (135.0 mM CsCl, 10.0 mM HEPES, 10.0 mM NaCl, 2.0 mM Mg-ATP, 0.3 mM Na-GTP, and 5.0 mM QX314) to detect sPSCs. Biocytin (1-3 mg/ml; Biotium) was dissolved in the pipette solution for post hoc identification of recorded iNs. Wholecell patch-clamp recordings were performed with a HEKA double patch-clamp EPC10 amplifier, with PatchMaster used for data acquisition. Data were analyzed offline with IgorPro and NeuroMatic (Rothman, J. S. & Silver, R. A. Front. Neuroinform. 12:14 (2018)). sPSCs were detected by NeuroMatic event detection, and each event was checked and identified as an sPSC manually. The identification of bursts was done manually. Bursts were characterized as a higher occurrence of sPSCs in a short time span followed by a period with a lower frequency of sPSCs.

### Biocytin Labeling.

Diffusion through gap junctions was visualized by filling of single iAs that were either grown on coverslips or implanted to the brains of adult mice. iAs on coverslips were grown as monocultures (described above).

For GFP+ iAs-SB that were implanted into adult mice, acute coronal brain slices were prepared. Mice were anesthetized and decapitated, brains were removed, and 250-μm coronal brain slices were prepared as previously described (Oki, K. et al. Stem Cells 30:1120-1133 (2012)). Coverslips and acute brain slices were constantly perfused with carbogenated ACSF. Individual iAs were patched with a potassium-gluconate-based intracellular solution (122.5 mM potassium gluconate, 12.5 mM KCl, 10.0 mM HEPES, 2.0 mM Na2ATP, 0.3 mM Na2-GTP, and 8.0 mM NaCl), using the whole-cell configuration for biocytin filling. Biocytin (1-3 mg/ml; Biotium) was dissolved in the pipette solution prior to recording. iAs were filled with biocytin for 10-15 min before the patch pipette was carefully removed and the cells were fixed with 4% PFA for 20 min or overnight for cells grown on coverslips or acute slices, respectively.

To detect biocytin-filled iAs grown on coverslips, Alexa Fluor 568-conjugated streptavidin (Thermo Fisher Scientific) diluted 1:500 in 0.025% TKPBS with 5% NDS was added for 1 h at RT.

For detection of biocytin-filled iAs-SB in acute brain slices, samples were rinsed three times for 10 min in KPBS, blocked for 1 h at RT with 1% TKPBS+10% NDS, incubated with primary antibody in blocking solution overnight at room temperature, rinsed three times for 10 min in 1% TKPBS, incubated for 2 h with secondary antibodies in 1% TKPBS+5% NDS, rinsed 3 times for 10 min in KPBS, placed on glass slides and dried overnight at room temperature before being mounted with DABCO:PVA and a coverslip.

### CRISPR Gene Editing.

hESCs were dissociated with Accutase, and 300,000 cells were transfected using FuGene (Active Motif), with 1 μg of each px462 Cas9n vector (Addgene #62987) carrying two different sgRNAs targeting the GFAP sequence near the site of the R239C mutation (Supplementary Table 7) and a 120-bp donor ssDNA carrying the R239C mutation and a C>T change in the PAM sequence of one of the sgRNAs (Supplementary Table 6). After transfection, cells were plated in MATRIGEL-coated six-well plates with mTeSR1 medium containing 10 μM ROCK inhibitor. Two days after transfection, a 48-h selection with 1.25 ng/ml of puromycin was performed. The surviving cells were dissociated after 7-10 days, and 100 cells were replated in six-well plates coated with laminin-521 (BioLamina), according to the manufacturer's instructions, with mTeSR1 medium containing 10 μM ROCK inhibitor. Subsequently, colonies were picked, plated, and expanded in a 24-well plate with mTesR1 medium containing 10 μM ROCK inhibitor. Clones were analyzed after DNA extraction with a DNeasy Blood and Tissue Kit (Qiagen) according to the manufacturer's instructions. Using specific primers (Table 7), we amplified a 279-bp region of the GFAP gene to check for incorporation of the mutation into the genome by sequencing. For off-target effects, genomic locations of interest were amplified with specific primers (Table 8). Sequence data were analyzed using SnapGene (GSL Biotech).

TABLE 7
GFAP genome editing oligonucleotides

| Oligonucleotide | Sequence |
|---|---|
| sgRNA 1 | 5′-ACTGCGTGCGGATCTCTTTC-3′ |
| sgRNA 2 | 5′-ACATGCATGAAGCCGAAGAG-3′ |
| Donor ssDNA | 5′-GTGGCCAAGCCAGACCTCACCGCAGCTCTG |
| | AAAGAGATCTGCACGCAGTATGAGGCAATGGCG |
| | TCCAGCAACATGCATGAAGCCGAAGAGTGGTAC |
| | CGCTCCAAGGTAGCCCTGCCTGTG-3′ |
| Primer Forward | 5′-CTGGTACCGCTTCTCTCACC-3′ |
| Primer Reverse | 5′-CAGCTTCTTCCACCCTCC-3′ |

TABLE 8
Primers for AxD genome editing off-target effects

| Primer name | Primer sequence |
|---|---|
| sgRNA A Chr11 Forward | 5′-TCCAGGCTATATGCCATTCC-3′ |
| sgRNA A Chr11 Reverse | 5′-TTACCCAGCAACTCATCTCTTT-3′ |
| sgRNA A Chr16 Forward | 5′-GCCCTTTACACCCTAACCCA-3′ |
| sgRNA A Chr16 Reverse | 5′-GCCCACACGGAGCTCTAA-3′ |
| sgRNA A Chr18 Forward | 5′-TGGTGATTGTCAAACATGCAGA-3′ |
| sgRNA A Chr18 Reverse | 5′-CAGTGAGCCGAGATTGCATC-3′ |
| sgRNA B Chr4 Forward | 5′-GCAACAGAGCAAGACACCAT-3′ |
| sgRNA B Chr4 Reverse | 5′-CTTTGCTTCATCTGCCGTGA-3′ |
| sgRNA B Chr10 Forward | 5′-AGGCATGGCAAAAGCAATAC-3′ |
| sgRNA B Chr10 Reverse | 5′-AAGTCCTCCCTGTTCACCAC-3′ |
| sgRNA B Chr20 Forward | 5′-TGTCCCTAGGTCTCCTGGTG-3′ |
| sgRNA B Chr20 Reverse | 5′-ATTTCCCCAGCTCTGACCTT-3′ |

### Implantation.

tetO-GFP cotransduced cells were dissociated with Accutase 7-10 days after induction, pelleted at 300 g for 5 min, and resuspended in Cytocon Buffer (43.25 g of myo-inositol, 5 g of polyvinyl alcohol, and 200 ml of PBS in 800 ml of distilled water) containing doxycycline (2.5 μg/ml) at 20,000-40,000 cells/μl. NSG mice at postnatal day 2-3 were anesthetized by hypothermia and placed on a chilled stereotaxic platform, and one unilateral injection of 1 μl was delivered with a 26-gauge Hamilton syringe into the forebrain at the following coordinates (from bregma and brain surface): anterior—posterior, 0-0.5 mm; medial-lateral, 1.5 mm; dorsal-ventral, −0.5-1.0 mm. Adult mice were anesthetized with isoflurane (3% during induction, 1.5-1.8% during maintenance) and placed into a stereotaxic frame. The mice received two injections of approximately 37,000-50,000 cells resuspended in 1 μl of Cytocon Buffer at the following coordinates: anterior-posterior, +0.38 mm; medial-lateral, ±1.5 mm; and dorsal-ventral −0.75 and −2.5 mm. For both newborn and adult implanted mice, 4-13 weeks after implantation, the mice were deeply anesthetized by intraperitoneal injection of sodium pentobarbital (100 mg per kg body weight) and perfused with saline (0.9% NaCl) followed by 4% PFA; the brains were removed and post-fixed overnight with 4% PFA at 4° C., cryoprotected in 30% sucrose cut into 30-μm coronal serial sections on a sliding microtome (Leica) and either processed for immunohistochemistry or stored in antifreeze solution at −20 ° C. All procedures were conducted in accordance with the European Union Directive and were approved by the ethical committee for the use of laboratory animals at Lund University and the Swedish Board of Agriculture.

### Data Presentation and Statistical Analyses.

Data are presented as the mean±s.e.m. unless otherwise stated in the respective figure legends. Statistical analyses (Prism) were performed using different tests as appropriate. For RT-qPCR results, unpaired two-tailed t-tests with Welch's correction for unequal variances were performed. For cytokine stimulation, statistical analysis was carried out by one-tailed ratio-paired t-test comparing iAs with and without treatment. For synapse formation, normal distribution was assessed with a Shapiro-Wilk test and an ordinary one-way ANOVA comparing all groups. In the case of AxD calcium imaging response, a multiple t-test was performed to compare WT and AxD iAs values at each time point. For the peak comparison, data did not show a normal distribution according to Shapiro-Wilk test, so an unpaired Mann-Whitney test was used. In the case of the electrophysiology experiments, a Mann-Whitney test or an unpaired t-test was used when appropriate. Significance was set at P<0.05.

### Example 9

### Step-By-Step Protocol for the Generation of iAs

### Reagents

DMEM/F-12 (ThermoFisher Scientific, cat. no. 31330038)
Neurobasal (ThermoFisher Scientific, cat. no. 21103049)
FBS (ThermoFisher Scientific, cat. no. 10082147)
N2 supplement (ThermoFisher Scientific, cat. no. 17502001)
B27 supplement (ThermoFisher Scientific, cat. no. 17504001)
NEAA (ThermoFisher Scientific, cat. no. 11140035)
GLUTAMAX (ThermoFisher Scientific, cat. no. 35050061)
Sodium Pyruvate (ThermoFisher Scientific, cat. no. 11360070)
bFGF (Peprotech, cat. no. 100-18B)
CNTF (Peprotech, cat. no. 450-13)
BMP4 (Peprotech, cat. no. 120-05ET)
dbcAMP (Sigma-Aldrich, cat. no. D0627-25MG)
N-acetyl-cysteine (Sigma-Aldrich, cat. no. A8199-10G)
Heparin-binding EGF-like growth factor (Sigma-Aldrich, cat. no. E4643-5OUG)
Polybrene (Sigma-Aldrich, cat. no. TR-1003-G)
Doxycycline (Sigma-Aldrich, cat. no. D9891-100UG)
ACCUTASE (ThermoFisher Scientific, cat. no. A1110501)
MATRIGEL (Corning, cat. no. 354230)
Rock inhibitor (StemCell Technologies, cat. no. Y-27632)
Thiazovivin (StemCell Technologies, cat. no. 72252)
Puromycin (ThermoFisher Scientific, cat. no. A1113803)
Hygromycin (ThermoFisher Scientific, cat. no. 10687010)

### Plasmids

GFAP::GFP (Gift from Chun-Li Zhang, University of Texas Southwestern)
pMD2.G (Addgene #12259)
pRSV-Rev (Addgene #12253)
pMDLg/pRRE (Addgene #12251)
M2-rtTA (Addgene #20342)
tetO-Sox9-Puro (generated in this work)
tetO.Nfia.Blast (generated in this work)
tetO.Nfib.Hygro (generated in this work)

### Reagent Setup

CaCl2: Dissolve at 2.5M in sterile water. Aliquot and store at −20° C.

Doxycycline: 25 mg/mL dissolved in water (10000× stock). Sterilize with a 0.22 μm filter and store at −20° C. Protect from light.

CNTF: Reconstitute at 10 μg/ml in sterile 10 mM sodium phosphate containing 0.1% BSA. Aliquot and store at −20° C.

bFGF: Reconstitute at 1 mg/ml in sterile 5 mM Tris, pH 7.6 containing 0.1% BSA. Aliquot and store at −20° C.

BMP4: Reconstitute at 10 μg/ml in sterile 4 mM HCl containing 0.1% BSA. Aliquot and store at −20° C.

dbcAMP: Reconstitute at 100 mg/ml in sterile water. Aliquot and store at −20° C. Protect from light.

N-acetyl-cysteine: Reconstitute at 50 mg/ml in sterile water. Aliquot and store at −20° C. Heparin-binding EGF-like growth factor: Reconstitute at 50 μg/ml in sterile PBS containing 0.1% BSA. Aliquot and store at −20° C.

### Cell Culture Medium

Expansion Medium:
DMEM/F-12
10% FBS
1% N2 supplement
1% Glutamax

FGF Medium:
Neurobasal
2% B27 supplement
1% NEAA
1% Glutamax
1% FBS
8 ng/ml FGF
5 ng/ml CNTF
10 ng/ml BMP4

Maturation Medium:
1:1 DMEM/F-12 and Neurobasal
1% N2
1% Sodium Pyruvate
1% Glutamax
5 μg/ml N-acetyl-cysteine
5 ng/ml heparin-binding EGF-like growth factor
10 ng/ml CNTF
10 ng/ml BMP4
500 μg/ml dbcAMP

### Equipment

Incubator BBD 6220 (ThermoFisher Scientific)
Centrifuge Rotina 420R (Hettich Lab Technology)
Water Bath GD100 (Grant Scientific)
Ultracentrifuge Beckman Optima L-100K (Beckman Coulter)

### Procedure

### Lentiviral Production

Lentiviruses were produced in HEK 293T cells by co-transfecting pMD2.G, pRSV-Rev and pMDLg/pRRE helper vectors together with the vector for one transcription factor using 2.5 M CaCl2. 75 μg of transcription factor plasmids was used together with 30 μg of PMDLg/pRRE, 22 μg of pMD2.G and 15 μg of pRSV-Rev plasmids for two T175 flasks. Medium was changed 16 hours after transfection and viruses were harvested 48 hours after transfection, pelleted by centrifugation (20,000×g for 2 hours at 4° C.) resuspended in 100 μl DMEM, aliquoted and kept at −80° C.

### iAs Generation

Day −2: Dissociate hESC or hiPSC with Accutase and replate 5×105 cells in MATRIGEL-coated 6-well plates with mTeSR1 containing 10 μM Rock inhibitor.

Day −1: Aspirate medium and add 2 ml of fresh mTeSR1 containing 8 μg/ml of Polybrene per well. Add 1 μl of each virus per well.

Day 0: Aspirate medium and add 2 ml of fresh mTeSR1 containing 2.5 μg/ml of Doxycycline per well.

Days 1 and 2: Aspirate medium and add 2 ml of Expansion Medium containing 2.5 μg/ml of Doxycycline, 1.25 μg/ml of Puromycin and 200 μg/ml of Hygromycin per well.

Day 3: Aspirate medium and add 2 ml of 3:4 of Expansion Medium and 1:4 of FGF Medium containing 2.5 μg/ml of Doxycycline and 200 μg/ml of Hygromycin per well.

Day 4: Aspirate medium and add 2 ml of 1:1 of Expansion Medium and FGF Medium containing 2.5 μg/ml of Doxycycline and 200 μg/ml of Hygromycin per well.

Day 5: Aspirate medium and add 2 ml of 1:4 of Expansion Medium and 3:4 of FGF Medium containing 2.5 μg/ml of Doxycycline and 200 μg/ml of Hygromycin per well.

Day 6: Aspirate medium and add 2 ml of FGF Medium containing 2.5 μg/ml of Doxycycline.

Day 7: Dissociate cells with Accutase and pellet at 300 g for 5 min. Re-plate cells in MATRIGEL-coated coverslips, petri dishes or wells according to desired output. Use the necessary amount of FGF Medium containing 2.5 μg/ml of Doxycycline.

Day 8: Aspirate medium and add fresh FGF Medium containing 2.5 μg/ml of Doxycycline.

Day 10: Aspirate half of the medium and add the same amount of Maturation Medium containing 2.5 μg/ml of Doxycycline. From here, change half of the medium every 2-3 days.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A method of converting human pluripotent cells into induced functional astrocytes (iAs) comprising:
contacting a population of human pluripotent stem cells with an effective dose of a reprograming system comprising Nfia or Nfib for a period of time sufficient to reprogram the pluripotent cells, wherein as a result of the method, a population of induced functional astrocytes is produced.

2. The method of claim 1, where the iAs exhibit functional characteristics of astrocytes.

3. The method of claim 1, wherein the human pluripotent stem cells overexpress Sox9.

4. The method of claim 1, wherein the human pluripotent stem cells are human embryonic stem cells (hESC).

5. The method of claim 1, wherein the human pluripotent stem cells are human induced pluripotent stem cells (hiPSC).

6. The method of claim 1, wherein the human pluripotent stem cells are genetically modified to carry a mutation associated with a neurological disease or disorder.

7. The method of claim 1 wherein the pluripotent stem cells are infected with one or more lentiviral vectors capable of conferring expression of one or more transcription factors selected from Nfia, Nfib, Nfia plus Sox9, Nfib plus Sox9, and Nfib plus Nfia plus Sox9, whereby the one or more transcription factors are overexpressed.

8. The method of claim 1, wherein expression of the one or more transcription factors is under control of a switchable promoter system.

9. The method of claim 7, wherein the switchable promoter system comprises a reverse tetracycline-controlled transactivator.

10. The method of claim 7, wherein the switchable promoter system is switched on for at least 7 days.

11. (canceled)

12. An isolated population of cells, produced by the method of claim 1, comprising at least 85% iAs.

13. (canceled)

14. The population of cells of claim 11, wherein the iAs are positive for at least one astrocyte biomarker.

15. The population of cells of claim 14, wherein the astrocyte biomarker is S100 calcium-binding protein B (S100B), Glial fibrillary acidic protein (GFAP), vimentin (VIM), Aldehyde Dehydrogenase 1 Family Member L1 (ALDH1L1), glutamate aspartate transporter (GLAST), CD44, Ki4.1, or any combination thereof.

16. The population of cells of claim 14, wherein the biomarker is S100B, GFAP, VIM, or any combination thereof.

17. The population of cells of claim 12, wherein the iAs have glycogen granules.

18. The population of cells of claim 12, wherein the iAs take up glutamate.

19. The population of cells of claim 12, wherein the iAs have increased intracellular calcium.

20. The population of cells of claim 12, wherein the iAs are capable of supporting synapse formation.

21. The population of cells of claim 12, wherein the iAs increase cytokine production in response to stimulation with IL-1β.

22. The population of cells of claim 12, wherein the iAs are capable of forming gap junctions. Preliminary Amendment Patent

23. The population of cells of claim 12, wherein the iAs are capable of surviving intracerebral implantation.


SEQUENCE LISTING (41 sequences)

SEQ ID NO: 1 - DNA, 27 bp, Artificial Sequence
  Sox9 forward primer
gaattcatgaatctcctggaccccttc

SEQ ID NO: 2 - DNA, 25 bp, Artificial Sequence
  Sox9 reverse primer
tctagagggtctggtgagctgtgtg

SEQ ID NO: 3 - DNA, 30 bp, Artificial Sequence
  Nfia forward primer
gaattccatgtattctccgctctgtctcac

SEQ ID NO: 4 - DNA, 27 bp, Artificial Sequence
  Nfia reverse primer
ggatccttatcccaggtaccaggactg

SEQ ID NO: 5 - DNA, 32 bp, Artificial Sequence
  Nfia forward primer
gaattcatgatgtattctcccatctgtctcac

SEQ ID NO: 6 - DNA, 25 bp, Artificial Sequence
  Nfib reverse primer
ggatccctagcccaggtaccaggac

SEQ ID NO: 7 - DNA, 24 bp, Artificial Sequence
  mNfib forward primer
gacatgaactctggtgtgaacctg

SEQ ID NO: 8 - DNA, 28 bp, Artificial Sequence
  mNfib reverse primer
gtagttggagaagacatatcttgatctc

SEQ ID NO: 9 - DNA, 24 bp, Artificial Sequence
  hNfib forward primer
gacatgaactcgggggtcaatctt

SEQ ID NO: 10 - DNA, 26 bp, Artificial Sequence
  hNfib reverse primer
gtagtcggagaagacatatcttgatc

SEQ ID NO: 11 - DNA, 21 bp, Artificial Sequence
  mSox9 forward primer
gcgagcactctgggcaatctc

SEQ ID NO: 12 - DNA, 18 bp, Artificial Sequence
  mSox9 reverse primer
ctgcccccctctgccaga

SEQ ID NO: 13 - DNA, 19 bp, Artificial Sequence
  hSox9 forward primer
cgagcactcggggcaatcc

SEQ ID NO: 14 - DNA, 18 bp, Artificial Sequence
  hSox9 reverse primer
ctgcccccctctggcaag

SEQ ID NO: 15 - DNA, 20 bp, Artificial Sequence
  GAPDH forward primer
gcaccgtcaaggctgagaac

SEQ ID NO: 16 - DNA, 19 bp, Artificial Sequence
  GAPDH reverse primer
agggatctcgctcctggaa

SEQ ID NO: 17 - DNA, 20 bp, Artificial Sequence
  sgRNA 1
actgcgtgcggatctctttc

SEQ ID NO: 18 - DNA, 20 bp, Artificial Sequence
  sgRNA 2
acatgcatgaagccgaagag

SEQ ID NO: 19 - DNA, 120 bp, Homo sapiens
gtggccaagccagacctcaccgcagctctgaaagagatctgcacgcagtatgaggcaatggcgtccagcaacatgcatgaagccgaagagtggtaccgctccaaggtagccctgcctgtg

SEQ ID NO: 20 - DNA, 20 bp, Artificial Sequence
  GFAP forward primer
ctggtaccgcttctctcacc

SEQ ID NO: 21 - DNA, 18 bp, Artificial Sequence
  GFAP reverse primer
cagcttcttccaccctcc

SEQ ID NO: 22 - DNA, 20 bp, Artificial Sequence
  sqRNA A CHr11 forward primer
tccaggctatatgccattcc

SEQ ID NO: 23 - DNA, 22 bp, Artificial Sequence
  sgRNA A Chr11 reverse primer
ttacccagcaactcatctcttt

SEQ ID NO: 24 - DNA, 20 bp, Artificial Sequence
  sgRNA A Chr 16 forward primer
gccctttacaccctaaccca

SEQ ID NO: 25 - DNA, 18 bp, Artificial Sequence
  sgRNA A Chr 16 reverse primer
gcccacacggagctctaa

SEQ ID NO: 26 - DNA, 22 bp, Artificial Sequence
  sgRNA A Chr18 forward primer
tggtgattgtcaaacatgcaga

SEQ ID NO: 27 - DNA, 20 bp, Artificial Sequence
  sgRNA A Chr18 reverse primer
cagtgagccgagattgcatc

SEQ ID NO: 28 - DNA, 20 bp, Artificial Sequence
  sgRNA B Chr4 forward primer
gcaacagagcaagacaccat

SEQ ID NO: 29 - DNA, 20 bp, Artificial Sequence
  sgRNA B Chr4 reverse primer
ctttgcttcatctgccgtga

SEQ ID NO: 30 - DNA, 20 bp, Artificial Sequence
  sgRNA B Chr10 forward primer
aggcatggcaaaagcaatac

SEQ ID NO: 31 - DNA, 20 bp, Artificial Sequence
  sgRNA B Chr10 reverse primer
aagtcctccctgttcaccac

SEQ ID NO: 32 - DNA, 20 bp, Artificial Sequence
  sgRNA B Chr20 forward primer
tgtccctaggtctcctggtg

SEQ ID NO: 33 - DNA, 20 bp, Artificial Sequence
  sgRNA B Chr20 reverse primer
atttccccagctctgacctt

SEQ ID NO: 34 - DNA, 24 bp, Artificial Sequence
  sgRNA 1
actgcgtgccggatctctttcagg

SEQ ID NO: 35 - DNA, 23 bp, Artificial Sequence
  sgRNA 2
acatgcatgaagccgaagagtgg

SEQ ID NO: 36 - DNA, 24 bp, Artificial Sequence
  sgRNA1
actgcgtgccggatctctttcagg

SEQ ID NO: 37 - DNA, 23 bp, Artificial Sequence
  sgRNA2
acatgcatgaagccgaagagtgg

SEQ ID NO: 38 - DNA, 96 bp, Artificial Sequence
  Altered GFAP Exon 4
agacctcaccgcagccctgaaagagatccgcacgcagtatgaggcaatggcgtccagcaacatgcatgaagccgaagagtggtaccgctccaaggt

SEQ ID NO: 39 - DNA, 121 bp, Artificial Sequence
  ssODN repair template
gtggccaagccagacctcaccgcagctctgaaagagatctgcacgcagtatgaggcaatggcgtccagcaacatgcaatgaagccgaagagtggtaccgctccaaggtagccctgcctgtg

SEQ ID NO: 40 - DNA, 54 bp, Artificial Sequence
  wild type GFAP sequence
aagccagacctcaccgcagccctgaaagagatccgcacgcagtatgaggcaatg

SEQ ID NO: 41 - DNA, 54 bp, Artificial Sequence
  mutated GFAP sequence
aagccagacctcaccgcagctctgaaagagatctgcacgcagtatgaggcaatg